(19) United States
(12) Patent Application Publication
(10) Pub. No.: US 2024/0001002 A1
(43) Pub. Date: Jan. 4, 2024

(54) PROTEIN HYDROGELS AND METHODS FOR THEIR PREPARATION

(71) Applicant: THE UNIVERSITY OF BRITISH COLUMBIA, Vancouver (CA)

(72) Inventors: HONGBIN LI, Vancouver (CA); LINGLAN FU, Vancouver (CA)

(73) Assignee: THE UNIVERSITY OF BRITISH COLUMBIA, Vancouver, BC (CA)

(21) Appl. No.: 18/037,287

(22) PCT Filed: Nov. 16, 2021
(86) PCT No.: PCT/CA2021/051622

§ 371 Date: May 16, 2023

Related U.S. Application Data
(60) Provisional application No. 63/115,174, filed on Nov. 18, 2020.

(51) Int. Cl. A61L 27/52; A61L 27/22

(52) U.S. Cl. CPC A61L 27/52; A61L 27/227; A61L 2430/06

(57) ABSTRACT

The present disclosure relates to methods of preparing protein hydrogels and to protein hydrogels which may, for example, be prepared from such methods. The methods comprise denaturing a protein in an aqueous environment to produce an aqueous composition comprising overlapping polypeptide chains; crosslinking the polypeptide chains to produce a denatured protein hydrogel comprising a crosslinked network of entangled polypeptide chains; and optionally at least partially renaturing the denatured protein hydrogel.

### CROSS-REFERENCE TO RELATED APPLICATIONS

The present disclosure claims the benefit of priority from co-pending U.S. provisional application No. 63/115,174 filed on Nov. 18, 2020, the contents of which are incorporated herein by reference in their entirety.

### FIELD

The present disclosure relates, for example, to protein hydrogels comprising a crosslinked network of entangled polypeptide chains and methods for their preparation.

### BACKGROUND

Many load-bearing tissues, ranging from muscle to cartilage, are protein-based biomaterials that exhibit finely regulated mechanical properties to uniquely suit their biological functions (Wainwright et al., 1982; Gosline et al., 2002). For example, load-bearing tissues, such as muscle and cartilage, exhibit mechanical properties that often combine high elasticity, high toughness and fast recovery, despite their different stiffness; 100 kPa for muscles and one to several MPa for cartilage (Wainwright et al., 1982; Higuchi, 1996; Linke et al., 1994; Hayes & Mockros, 1971; Temple et al., 2016; Williamson et al., 2003; Kerin et al., 1998). The advances in protein engineering and protein mechanics have made it possible to engineer protein-based biomaterials to mimic soft load-bearing tissues, such as muscles (Lv et al., 2010; Wu et al., 2018; Khoury et al., 2018). For example, to engineer biomimetics of these biological tissues, protein-based hydrogels have been widely explored (Li et al., 2020). Protein hydrogels are generally soft, with a Young's modulus up to about 100 kilopascal (kPa) (Lv et al., 2010; Elvin et al., 2005). Thus, current protein hydrogel technologies have achieved considerable success in mimicking softer tissues (Li et al., 2020; Elvin et al., 2005; McGann et al., 2013), such as muscle (Lv et al., 2010; Wu et al., 2018; Fang et al., 2013).

Stiff biological tissues, such as cartilage, tendons and ligaments, often integrate seemingly mutually incompatible mechanical properties into themselves (Wainwright et al., 1982). Mimicking such properties using synthetic hydrogels has been challenging. It often occurs that optimizing one property is at the expense of another one. To alleviate this issue and achieve high stiffness and high toughness, polymer hydrogels of designed network structures and polymer composite hydrogels have been developed (Gong et al., 2003; Gong et al., 2010; Xu et al., 2019; Okumura et al., 2001; Bin Imran et al., 2014; Liu et al., 2017; Wang et al., 2012; Sun et al., 2020), such as double network hydrogels (Gong et al., 2003; Gong et al., 2010), co-joined network hydrogels (Xu et al., 2019) and slide-ring hydrogels (Okumura et al., 2001; Bin Imran et al., 2014). Sacrificial bonds/weak secondary network that can be ruptured are often introduced into the hydrogel as an energy dissipation mechanism (Gong et al., 2010; Sun et al., 2012; Zhao, 2014). Although high stiffness and high toughness have been achieved in some of these hydrogels, slow recovery and mechanical fatigue are often present, due to the irreversible rupture of these sacrificial bonds and/or slow dynamics of weak secondary networks.

As mentioned hereinabove, it is challenging to engineer protein biomaterials to achieve the mechanical properties exhibited by stiff tissues, such as articular cartilage (Williamson et al., 2003; Mohan et al., 2009), or to develop stiff synthetic extracellular matrices for cartilage stem/progenitor cell differentiation (Jiang & Tuan, 2015). Stiffer tissues often have a modulus on the order of megapascal (MPa) and bear tensile as well as compressive loads, making them challenging to achieve for the current protein hydrogel technology. For example, articular cartilage is a superb load-bearing material made of collagen and proteoglycans. It has a modulus ranging from one to several MPa, can withstand a load up to a hundred MPa and sustain millions of cycles of loading-unloading without much fatigue, showing fast recovery in its shape and mechanical properties (Wainwright et al., 1982; Kern et al., 1998; McCutchen, 1978). Engineering protein hydrogels that can combine these seemingly mutually incompatible properties (high stiffness, high toughness and fast recovery) remains challenging.

Chain entanglement is an important strengthening mechanism in polymeric materials (Treloar, 1975). Due to their long length, polymer chains in the network can cross each other, resulting in chain entanglement. Chain entanglement effectively increases the crosslinking density in the polymer network and leads to improved Young's modulus (Treloar, 1975). However, in muscle fibers, the muscle protein titin is organized as parallel bundles without chain entanglement (Higuchi, 1996; Linke et al., 1994). In the engineered muscle mimetic protein hydrogels constructed from tandem modular protein-based elastomeric proteins, no chain entanglement is present either, due to the short contour length of such elastomeric proteins; about 10 to about 40 nm in length (Lv et al., 2010; Fang et al., 2013).

### SUMMARY

This disclosure is based in part on the fortuitous discovery that the mechanical properties of cross-linked protein hydrogels can be significantly improved if the native protein is denatured prior to chemical cross-linking. A new denatured crosslinking hydrogelation approach is disclosed herein which combines forced-unfolding of proteins and chain entanglement, and may, for example, enable the engineering of strong and tough protein hydrogels. On the one hand, forced-unfolding of proteins provides an efficient mechanism for energy dissipation, and the ability to refold allows the hydrogel to recover its mechanical properties rapidly and minimize mechanical fatigue. On the other hand, chain entanglement allows the hydrogel to achieve high stiffness. In so doing, these effects work cooperatively to allow the integration of high stiffness, high toughness, fast recovery and high compressive strength into protein hydrogels. Although the engineered protein hydrogel has a single network structure, its superb mechanical properties essentially converted a muscle-like soft biomaterial to a stiff material exhibiting mechanical properties that mimic cartilage.

Accordingly, the present disclosure includes a method of preparing a protein hydrogel, the method comprising:
denaturing a protein in an aqueous environment to produce an aqueous composition comprising overlapping polypeptide chains;
crosslinking the polypeptide chains to produce a denatured protein hydrogel comprising a crosslinked network of entangled polypeptide chains; and
optionally at least partially renaturing the denatured protein hydrogel to produce a renatured protein hydrogel comprising a crosslinked network of entangled polypeptide chains.

In an embodiment, the denaturing comprises subjecting the protein to a chaotropic agent. In another embodiment, the aqueous environment comprises the chaotropic agent and the method comprises introducing the protein into the aqueous environment. In a further embodiment, the chaotropic agent comprises guanidinium chloride. In another embodiment, the concentration of the guanidium chloride in the aqueous environment is in the range of from about 6 M to about 8M. In another embodiment, wherein the concentration of the guanidium chloride in the aqueous environment is about 7M.

In an embodiment, the concentration of the protein in the aqueous environment is about 20% (w/v).

In an embodiment, the method comprises the at least partial renaturing of the denatured protein hydrogel to produce the renatured protein hydrogel.

In an embodiment, the renaturing comprises equilibrating the denatured protein hydrogel in phosphate buffered saline.

In an embodiment, the crosslinking is carried out in a mold.

In an embodiment, the protein is a globular protein. In another embodiment, the protein is a tandem modular protein. In a further embodiment, the protein has a molecular weight of greater than 33 kDa. In another embodiment, the protein has greater than 300 residues. In an embodiment, the protein is an engineered protein. In another embodiment, the protein comprises ferredoxin-like folds. In a further embodiment, the protein comprises (FL)ₓ, (FL-M23C)ₓ, (NuG2)ₓ, (GB1)ₓ, (GA)ₓ, where x is the number of protein repeat units and x is at least 4, GRG₅RG₄R, N₄RN₄RNR or combinations thereof. In another embodiment, the protein comprises (FL)₈.

The present disclosure also includes a protein hydrogel prepared by a method as described herein.

The present disclosure also includes a protein hydrogel comprising a crosslinked network of entangled polypeptide chains.

In an embodiment, the crosslinked network of entangled polypeptide chains comprises a combination of folded domains and unfolded domains. In another embodiment, the crosslinked network of entangled polypeptide chains comprises about 50% folded domains.

In an embodiment, the concentration of the crosslinked network of entangled polypeptide chains in the protein hydrogel is about 20% (w/v).

In an embodiment, the crosslinked network of entangled polypeptide chains is derived from a globular protein. In another embodiment, the crosslinked network of entangled polypeptide chains is derived from a tandem modular protein. In a further embodiment, the crosslinked network of entangled polypeptide chains is derived from a protein having molecular weight of greater than about 33 kDa. In another embodiment, the crosslinked network of entangled polypeptide chains is derived from a protein having greater than 300 residues. In an embodiment, the crosslinked network of entangled polypeptide chains is derived from an engineered protein. In another embodiment, the crosslinked network of entangled polypeptide chains is derived from a protein comprising ferredoxin-like folds. In a further embodiment, the crosslinked network of entangled polypeptide chains is derived from a protein comprising (FL)ₓ, (FL-M23C)ₓ, (NuG2)ₓ, (GB1)ₓ, (GA)ₓ, where x is the number of protein repeat units and x is at least 4, GRG₅RG₄R, N₄RN₄RNR or combinations thereof. In another embodiment, the crosslinked network of entangled polypeptide chains is derived from a protein comprising (FL)₈.

In another aspect, the present disclosure provides a method of improving the mechanical properties of a protein hydrogel wherein: a) the protein is denatured; b) the denatured protein is chemically or photochemically cross-linked; c) the cross-linked protein is optionally allowed to at least partially renature. In another aspect, the protein is a globular protein. In yet another aspect, the protein is a tandem modular protein. In some aspects, the protein has a molecular weight of more than about 33 kDa, or more than about 300 residues. In some aspects, the protein is (FL)ₓ, (NuG)ₓ, (GB1)ₓ, (GA)ₓ, where x is the number of protein repeat units and x may be at least 4. In some aspects, the protein is Bovine Serum Albumin, GRG₅RG₄R, or N₄RN₄RNR. In one aspect, the protein is (FL)₈. In one embodiment, the improved protein hydrogel has a Young's modulus at least about 3.5 times higher than a hydrogel made directly from the native protein. In other aspects, the Young's modulus may be improved at least about 5 times, or about 10 times, or about 30 times. In one embodiment, the improved protein hydrogel has a compressive modulus at least 5 times higher than a hydrogel made directly from the native protein. In other aspects, the compressive modulus may be improved at least about 10 times, or about 20 times, or about 40 times. In one embodiment, the improved protein hydrogel has a toughness at least 5 times higher than a hydrogel made directly from the native protein. In one embodiment, the improved protein hydrogel has a breaking stress under compression at least 300 times higher than a hydrogel made directly from the native protein. In one embodiment, the improved protein hydrogel has a compressive modulus at least 5 times higher than a hydrogel made directly from the native protein. In other aspects, the compressive modulus may be improved at least about 10 times, or about 20 times, or about 40 times.

In another aspect, the present disclosure provides a protein hydrogel composition that is created by a method comprising: a) denaturing the protein; b) chemically or photochemically cross-linking the protein; c) optionally allowing the protein to at least partially renature. In another aspect, the protein is a globular protein. In yet another aspect, the protein is a tandem modular protein. In some aspects, the protein has a molecular weight of more than about 33 kDa, or more than about 300 residues. In some aspects, the improved protein hydrogel composition is created from (FL)ₓ, (NuG)ₓ, (GB1)ₓ, (GA)ₓ, where x is the number of protein repeat units and x may be at least 4. In some aspects, the protein hydrogel composition is created from Bovine Serum Albumin, GRG₅RG₄R, or N₄RN₄RNR. In one aspect, the improved protein hydrogel is created from (FL)₈. In one embodiment, the improved protein hydrogel composition has a Young's modulus at least 3.5 times higher than a hydrogel made directly from the native protein. In other aspects, the Young's modulus may be improved at least about 5 times, or about 10 times, or about 30 times. In one embodiment, the improved protein hydrogel composition has a compressive modulus at least 300 times higher than a hydrogel made directly from the native protein.

In one aspect, the present disclosure provides the use of a protein hydrogel composition as described herein for a use as support material or scaffold in a biological context. In a further aspect, the protein hydrogel composition may be an artificial cartilage material. In a further aspect, the protein hydrogel composition may be used as a scaffold for a method of treatment of a subject having a disorder characterized by tissue damage or loss, said tissue consisting of articular cartilage or bone tissue, the method comprising a) implanting the scaffold in the subject to thereby induce formation of the tissue and treat the disorder characterized by tissue damage. In a further embodiment, the protein hydrogel composition can be used for the treatment of subjects with defects in cartilage produced through injury or disease. Defects due to injury can be sports or accident-related or due to repetitive use. Defects due to disease include those resulting from osteoarthritis and rheumatoid arthritis. In yet another aspect, the protein hydrogel composition may be used to treat a subject by repairing or replacing cartilage in a load-bearing joint such as a knee, wrist, ankle, shoulder, spine or hip. The protein hydrogel compositions disclosed herein may be used or applied for non-medical benefits or applications such as soft actuators and soft grippers for soft robotics.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should rather be given the broadest interpretation consistent with the description as a whole.

### BRIEF DESCRIPTION OF THE DRAWINGS

FIG. 1 is a plot showing representative force-distance curves of ferredoxin-like globular protein FL domain at a pulling speed of 50 nm/s. The inset is a schematic showing the three-dimensional structure of FL (PDB code: 2KL8).

FIG. 2 shows schematics of the NC-(FL)₈ hydrogels and their preparation according to a comparative example of the present disclosure.

FIG. 3 shows physical entanglements enhanced the stiffness of the (FL)₈ hydrogels; photographs of both hydrogels after being equilibrated in 7M GdHCl, wherein the D-DC (Denatured-Denatured Crosslinking) hydrogel is self-standing and swells to a much less degree than the D-NC (Denatured-Native Crosslinking) hydrogel (top); and stress-strain curves of D-DC (*) and D-NC (**) (FL)₈ hydrogels (200 mg/mL) with an inset that is the zoom view of the stress-strain curve of the D-NC hydrogel, wherein the D-DC hydrogel showed a Young's modulus of about 50 kPa, which is significantly higher than that of D-NC hydrogel which had a Young's modulus of about 1 kPa (bottom).

FIG. 4 shows fluorescence spectra of acid hydrolyzed 20% D-DC and D-NC (FL)₈ hydrogels prepared from the same weight of lyophilized (FL)₈ proteins. Fluorescence at 410 nm resulted from the dityrosine fluorescence.

FIG. 5 shows a photograph of N-DC (Native-Denatured crosslinking) (left) and N-NC (Native-Native Crosslinking) (right) hydrogels equilibrated in PBS, wherein the N-DC hydrogel is translucent, while N-NC hydrogel is opaque (top); and stress-strain curves of N-DC (*) and N-NC (**) (FL)₈ hydrogels (200 mg/mL), with an inset that is the zoom view of the stress-strain curve of the N-NC hydrogel, wherein the N-DC hydrogel showed a Young's modulus of about 0.7 MPa, which is significantly higher than that of N-NC hydrogel (about 20 kPa) and the N-DC hydrogel ruptured at about 100% strain (bottom).

FIG. 6 shows a plot of swelling ratios showing that DC (FL)₈ hydrogels can be cycled between the N-DC and D-DC states reversibly.

FIG. 7 shows photographs of scanning electron microscopy imaging of the N-DC (top) and N-NC (bottom) (FL)₈ hydrogels. Both hydrogels showed porous network structures, however, the pore size of the N-DC hydrogel (about 2 μm) is significantly smaller than that of the N-NC hydrogel (about 20 μm). Scale bars in each image show 50 μm.

FIG. 8 shows schematics of the chain entangled network structure of the N-DC (FL)₈ hydrogel and its preparation according to an example of the present disclosure.

FIG. 9 shows typical tensile stress-strain curves of 20% N-DC (FM23C)₈ hydrogels. Inset shows an optical photograph of the N-DC (FM23C)₈ hydrogel.

FIG. 10 shows photographs of (FL-M23C)₈ N-DC hydrogels; a (FL-M23C)₈ N-DC hydrogel under UV illumination light, wherein the blue (observable in color images) fluorescence was from the dityrosine crosslinking points (top); and a (FL-M23C)₈ N-DC hydrogel under UV-illumination after labeling with IAEDANS, wherein the cyan (observable in color images) fluorescence was from the labeling of the exposed cysteine residues, and indicated that some FL domains were unfolded in the hydrogel (bottom).

FIG. 11 shows a fluorescence spectrum of 5-((2-((iodoacetyl)amino)ethyl)amino) naphthalene-1-sulfonic acid (IAEDANS) labeled 20% (FL-M23C)₈ hydrogel wherein dotted lines are Gaussian fits to the two fluorescence peaks, one is the dityrosine fluorescence at 410 nm, and the other one is the IAEDANS fluorescence at 490 nm.

FIG. 12 shows mechanical properties of N-DC and D-DC (FL)₈ hydrogels in tensile testing: Young's modulus and breaking strain of N-DC and D-DC (FL)₈ hydrogels. It is evident that the N-DC hydrogel exhibited much higher Young's modulus than the N-NC hydrogel.

FIG. 13 shows tensile properties of N-DC (FL)₈ hydrogels at different protein concentrations (10%, 15% and 20%) and (FL)₁₆ hydrogels (20%): Young's modulus (top left); breaking strain (top right); toughness (bottom left); and swelling ratio (bottom right).

FIG. 14 shows stretching-relaxation stress-strain curves of the N-DC (FL)₈ hydrogel, wherein a large hysteresis was present in the stretching and relaxation curves, indicative of large energy dissipation (top); and toughness and swelling ratio of N-DC and D-DC (FL)₈ hydrogels, wherein it is evident that the N-DC hydrogel exhibited higher toughness than the N-NC hydrogel (bottom).

FIG. 15 shows the hysteresis between stretching and relaxation curves can be recovered rapidly: the hydrogel was first stretched to about 60% strain and then relaxed to zero strain, after waiting for certain time Δt, the hydrogel was subject to the stretching-relaxation cycle again and the hysteresis recovery can be directly observed (top); and the kinetics of the hysteresis recovery in N-DC (FL)₈ hydrogel: about 70% of the hysteresis can be recovered rapidly within a few seconds, and the remaining 30% hysteresis can be recovered following a double-exponential kinetics, a red line (observable in a color image) is a double exponential fit to the data, with a rate constant k₁ of 0.05±0.02 s⁻¹ and k₂ of (1.7±0.3)×10⁻³ s⁻¹, respectively (bottom).

FIG. 16 shows exemplary photographs showing that the N-DC (FL)₈ hydrogel can resist cutting with a sharp scalpel: initial state (top); cut (middle); and relax (bottom). Scale bar in top image shows 5 mm.

FIG. 17 shows compressive stress-strain curves of the N-DC (*) and N-NC (FL)₈ (**) hydrogels. Inset is a zoom view of the stress-strain curves of N-NC hydrogel. The N-DC hydrogel can be compressed to more than 80% strain and sustain a compressive stress of >70 MPa without failure. A large hysteresis was present between the loading and unloading curves, indicating that a large amount of energy was dissipated.

FIG. 18 shows exemplary photographs of the N-DC (FL)₈ hydrogel in its initial state (left); under compression (center top, center bottom); and after unloading, wherein the hydrogel recovered its shape rapidly (right top, right bottom).

FIG. 19 shows stress-strain curves of a N-DC (FL)₈ hydrogel compressed to failure. Insets show the photographs of the hydrogel right after failure (1^{st} cycle; left) and after three more consecutive compression-unloading cycles (4^{th} cycle; right). Cracks were observed right after the failure. Subsequent compression led to the propagation of the crack.

FIG. 20 shows a consecutive compression-unloading curve of the N-DC hydrogel. The hysteresis grows with the increasing of the strain. The toughness of the hydrogel was about 3.2 MJ/m³. The inset is a zoom view of the stress-strain curves at lower strain.

FIG. 21 shows consecutive compression-unloading cycles show that the hysteresis of the N-DC hydrogel can be recovered rapidly. The inset shows the hysteresis recovery kinetics of the hydrogel. About 65% hysteresis can be recovered right after unloading, and the rest of the hysteresis can be recovered following a double exponential kinetics, with k₁ of 0.10±0.02 s⁻¹ and k₂ of (2.0±0.3)×10⁻³ s⁻¹.

FIG. 22 shows consecutive loading-unloading curves of the N-DC (FL)₈ hydrogel at a frequency of about 0.37 Hz. The pulling speed was 100 mm/min. In each cycle, the hydrogel was stretched to 60% strain and subsequently relaxed to zero strain. After 100 cycles, the hydrogel displayed little fatigue, and the stress of the hydrogel at 60% strain retained about 90% of the original stress in the first cycle.

FIG. 23 shows consecutive compression-unloading curves of a N-DC (FL)₈ hydrogel at a frequency of 0.08 Hz (top) and 0.67 Hz (bottom). The loading rate was 20 mm/min (top) and 200 mm/min (bottom), respectively.

### DETAILED DESCRIPTION

### I. Definitions

Unless otherwise indicated, the definitions and embodiments described in this and other sections are intended to be applicable to all embodiments and aspects of the disclosure herein described for which they would be understood to be suitable by a person skilled in the art. Although various embodiments and aspects of the disclosure are disclosed herein, many adaptations and modifications may be made within the scope of the disclosure in accordance with the common general knowledge of those skilled in this art. Such modifications include the substitution of known equivalents for any embodiment or aspect of the disclosure in order to achieve the same result in substantially the same way.

Numeric ranges are inclusive of the numbers defining the range.

Terms of degree such as “about” and “approximately” as used herein mean a reasonable amount of deviation of the modified term such that the end result is not significantly changed. These terms of degree should be construed as including a deviation of at least ±5% of the modified term if this deviation would not negate the meaning of the term it modifies.

As used herein, the words “comprising” (and any form thereof, such as “comprise” and “comprises”), “having” (and any form thereof, such as “have” and “has”), “including” (and any form thereof, such as “include” and “includes”) or “containing” (and any form thereof, such as “contain” and “contains”), are inclusive or open-ended and do not exclude additional, unrecited elements or process/method steps. As used herein, the word “consisting” and its derivatives are intended to be close-ended terms that specify the presence of the stated features, elements, components, groups, integers and/or steps, and also exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of these features, elements, components, groups, integers and/or steps.

As used herein, the singular forms “a”, “an” and “the” include plural referents unless the context clearly dictates otherwise. Thus, for example, reference to “a thing” includes more than one such thing unless the context clearly dictates otherwise.

The term “suitable” as used herein means that the selection of the particular compound, material and/or conditions would depend on the specific synthetic manipulation to be performed, and/or the identity of the compound(s) to be transformed, but the selection would be well within the skill of a person skilled in the art. All method steps described herein are to be conducted under conditions sufficient to provide the product shown.

The expression “proceed to a sufficient extent” as used herein with reference to the reactions or method steps disclosed herein means that the reactions or method steps proceed to an extent that conversion of the starting material or substrate to product is maximized. Conversion may be maximized when greater than about 5, 10, 15, 20, 25, 30, 35, 40, 45, 50, 55, 60, 65, 70, 80, 85, 90, 95 or 100% of the starting material or substrate is converted to product.

### II. Methods

A new denatured crosslinking hydrogelation approach is disclosed herein which combines forced-unfolding of proteins and chain entanglement, and may, for example, enable the engineering of strong and tough protein hydrogels.

Accordingly, the present disclosure includes a method of preparing a protein hydrogel, the method comprising:
denaturing a protein in an aqueous environment to produce an aqueous composition comprising overlapping polypeptide chains;
crosslinking the polypeptide chains to produce a denatured protein hydrogel comprising a crosslinked network of entangled polypeptide chains; and
optionally at least partially renaturing the denatured protein hydrogel to produce a renatured protein hydrogel comprising a crosslinked network of entangled polypeptide chains.

The denaturing can comprise any suitable method, the selection of which can be made by a person skilled in the art. For example, it would be appreciated by a person skilled in the art that in the methods of the present disclosure, the method of denaturing the protein unfolds the protein thereby producing polypeptide chains that can overlap in the aqueous composition. In some embodiments of the present disclosure, the method of denaturing is desirably reversible, such that, for example, a denatured protein hydrogel can be at least partially renatured to produce a renatured protein hydrogel.

In an embodiment, the denaturing comprises subjecting the protein to a chaotropic agent. The term “chaotropic agent” as used herein refers to an agent that is capable of disrupting the hydrogen bonding network between water molecules and thereby reduces the stability of the native state of the protein by weakening the hydrophobic effect such that the protein is unfolded to produce polypeptide chains that overlap in the aqueous environment of the methods of the present disclosure. In an embodiment, the aqueous environment comprises the chaotropic agent and the method comprises introducing the protein into the aqueous environment. The chaotropic agent is any suitable chaotropic agent. In an embodiment, the chaotropic agent comprises, consists essentially of or consists of guanidinium chloride. In another embodiment, the chaotropic agent comprises guanidinium chloride. In a further embodiment, the chaotropic agent consists essentially of guanidium chloride. In another embodiment, the chaotropic agent consists of guanidium chloride. The concentration of the chaotropic agent is any suitable concentration. For example, a person skilled in the art would appreciate that at high concentrations of guanidium chloride (e.g. about 6M or greater), proteins typically lose their ordered structure which may, for example, produce polypeptide chains suitable for overlapping in the aqueous environment of the methods of the present disclosure. Accordingly, in an embodiment, the concentration of the guanidium chloride in the aqueous environment is in the range of from about 6M to about 8M. In another embodiment, the concentration of the guanidium chloride in the aqueous environment is about 7M.

The concentration of the protein in the aqueous environment is selected such that the polypeptide chains produced from the denaturation of the protein overlap in the aqueous environment. In an embodiment, the concentration of the protein in the aqueous environment is at least 5% (w/v). In another embodiment, the concentration of the protein in the aqueous environment is at least 15% (w/v). In another embodiment, the concentration of the protein in the aqueous environment is from about 15% (w/v) to about 25% (w/v). In another embodiment, the concentration of the protein in the aqueous environment is about 20% (w/v).

In some embodiments, the method comprises the at least partial renaturing of the denatured protein hydrogel to produce the renatured protein hydrogel. The renaturing can comprise any suitable method, the selection of which can be made by a person skilled in the art, and may, for example, depend on the method of denaturing. In an embodiment, the renaturing comprises equilibrating the denatured protein in an aqueous composition comprising sodium chloride (e.g. an approximately physiological concentration of sodium chloride) and optionally having a buffer to maintain the aqueous composition at an approximately physiological pH (e.g. a pH of about 7 or from 7.35 to 7.45 or 7.4). In an embodiment, the aqueous composition comprises phosphate buffered saline (e.g. an aqueous composition comprising 137 mM NaCl, 2.7 mM KCl, 10 mM Na₂HPO₄, and 1.8 mM NaH₂PO₄), Tris-buffered saline or 4-(2-hydroxyethyl)-1-piperazineethanesulfonic acid (HEPES)-buffered saline. In an embodiment, the aqueous composition comprises phosphate buffered saline. In another embodiment, the renaturing comprises equilibrating the denatured protein hydrogel in phosphate buffered saline. The renaturing is carried out for a time and under conditions for the at least partial renaturing of the denatured protein hydrogel to the renatured protein to proceed to a sufficient extent. For example, in an embodiment, the denatured protein hydrogel is contacted with the phosphate buffered saline for a time of from about 8 hours to about 3 days or about 24 hours at ambient temperature such as a temperature of about 4° C. to about 40° C. or about 25° C.

Methods of crosslinking proteins are well known in the art and the methods of the present disclosure can comprise any suitable method of crosslinking, chemical or photochemical. The term “photochemical crosslinking” as used herein refers to methods comprising light irradiation to activate a photoreactive group involved in a chemical reaction to crosslink the polypeptide chains. While the term “chemical crosslinking” may also include “photochemical crosslinking”, the skilled person will appreciate that in certain embodiments herein, for example, wherein it is referred to as an alternative to “photochemical crosslinking” it refers to non-photochemical crosslinking methods such as cysteine-specific crosslinking methods (i.e. methods comprising the use of thiol-reactive reagents to crosslink the polypeptide chains), lysine-specific crosslinking methods (i.e. methods comprising the use of amine-reactive reagents to crosslink the polypeptide chains) and enzymatic crosslinking methods (i.e. methods comprising the use of an enzyme to crosslink the polypeptide chains). In some embodiments, the crosslinking is carried out in a mold. For example, in an embodiment, the aqueous composition comprising overlapping polypeptide chains is introduced into a suitable mold (e.g. a mold comprising plexiglass), and subjected to crosslinking for a time for the crosslinking of the polypeptide chains to produce the denatured protein hydrogel to proceed to a sufficient extent. In an embodiment, the method further comprises removing the denatured protein hydrogel from the mold. The conditions for the crosslinking such as the time and/or the temperature may depend, for example, on the method of crosslinking but can be readily selected by a person skilled in the art.

The protein is any suitable protein. For example, in the examples of the present disclosure, protein hydrogels were constructed from a range of proteins. In an embodiment, the protein is a globular protein. In another embodiment, the protein is a tandem modular protein. It will be appreciated by a person skilled in the art that the protein is capable of producing polypeptide chains of a length suitable for overlapping in the aqueous environment. In an embodiment, the protein has a molecular weight of greater than 33 kDa. In another embodiment, the protein has greater than 300 residues. In an embodiment, the polypeptide chains have a length of at least about 100 nm or at least about 200 nm. In another embodiment, the polypeptide chains have a length of about 260 nm.

In an embodiment, the protein is an engineered protein. The term “engineered protein” as used herein refers to a polypeptide that does not occur in nature. For example, in an embodiment, the engineered protein comprises at least one change, such as an addition, deletion and/or substitution relative to a naturally occurring polypeptide, wherein such at least one change is introduced by recombinant DNA techniques. In another embodiment, the engineered protein comprises an amino acid sequence generated by man, an artificial protein, a fusion protein or a chimeric polypeptide. Methods of preparing engineered proteins are well known in the art and the selection of a suitable method or source such as a commercial source for a desired engineered protein can be readily made by the skilled person.

In an embodiment, the protein comprises ferredoxin-like folds. The term “ferredoxin-like folds” as used herein in reference to a protein refers to a motif comprising a topology of 2 α helices and 4 β strands with a βαββαβ secondary structure such that the two terminal β strands hydrogen-bond to the central two β-strands, forming a four-stranded, antiparallel β-sheet covered on one side by two α-helices.

In an embodiment, the protein comprises, consists essentially of or consists of (FL)ₓ, (FL-M23C)ₓ, (NuG2)ₓ, (GB1)ₓ, (GA)ₓ, where x is the number of protein repeat units and x is at least 4, GRG₅RG₄R, N₄RN₄RNR or combinations thereof. In another embodiment, the protein comprises (FL)ₓ, (FL-M23C)ₓ, (NuG2)ₓ, (GB1)ₓ, (GA)ₓ, where x is the number of protein repeat units and x is at least 4. In an embodiment, each x is independently an integer of from 4 to 20. In another embodiment, each x is independently an integer of from 4 to 16. In another embodiment, each x is independently an integer of from 4 to 12. In a further embodiment, x is 8. In another embodiment, x is 16. In another embodiment, the protein comprises, consists essentially of or consists of GRG₅RG₄R or N₄RN₄RNR. In an embodiment, the protein comprises, consists essentially of or consists of (FL)₈, (FL)₁₆, (FL-M23C)₈, (NuG2)₈, (GB1)₈ or (GA)₈. In another embodiment, the protein comprises, consists essentially of or consists of (FL)₈. In a further embodiment, the protein comprises (FL)₈. In another embodiment, the protein consists essentially of (FL)₈. In another embodiment, the protein consists of (FL)₈. In an embodiment, the protein comprises, consists essentially of or consists of (FL)₁₆. In another embodiment, the protein comprises, consists essentially of or consists of (FL-M23C)₈. In a further embodiment, the protein comprises, consists essentially of or consists of (NuG2)₈. In an embodiment, the protein comprises, consists essentially of or consists of (GB1)₈. In another embodiment, the protein comprises, consists essentially of or consists of (GA)₈.

### III. Protein Hydrogels

A new denatured crosslinking hydrogelation approach is disclosed herein which combines forced-unfolding of proteins and chain entanglement, and may, for example, enable the engineering of strong and tough protein hydrogels. Although the engineered protein hydrogels had a single network structure, the superb mechanical properties of the protein hydrogels comprising chain entanglements essentially converted a muscle-like soft biomaterial to a stiff material exhibiting mechanical properties that mimic cartilage.

Accordingly, the present disclosure includes a protein hydrogel comprising a crosslinked network of entangled polypeptide chains. In an embodiment, the protein hydrogel is prepared by a method of preparing a protein hydrogel of the present disclosure.

In an embodiment, the crosslinked network of entangled polypeptide chains comprises a combination of folded domains and unfolded domains. In another embodiment, the crosslinked network of entangled polypeptide chains comprises about 50% folded domains.

In an embodiment, the concentration of the crosslinked network of entangled polypeptide chains in the protein hydrogel is at least 5% (w/v). In another embodiment, the concentration of the crosslinked network of entangled polypeptide chains in the protein hydrogel is at least 15% (w/v). In another embodiment, the concentration of the crosslinked network of entangled polypeptide chains in the protein hydrogel is from about 15% (w/v) to about 25% (w/v). In another embodiment, the concentration of the crosslinked network of entangled polypeptide chains in the protein hydrogel is about 20% (w/v).

Methods of crosslinking proteins are well known in the art and the protein hydrogels of the present disclosure can comprise any suitable crosslinks, chemical or photochemical.

The crosslinked network of entangled polypeptide chains can be derived from any suitable protein. For example, in the examples of the present disclosure, protein hydrogels were constructed from a range of proteins. In an embodiment, the crosslinked network of entangled polypeptide chains is derived from a globular protein. In another embodiment, the crosslinked network of entangled polypeptide chains is derived from a tandem modular protein. It will be appreciated by a person skilled in the art that the protein is capable of producing polypeptide chains of a length suitable for entanglement in the protein hydrogels. In an embodiment, the crosslinked network of entangled polypeptide chains is derived from a protein having molecular weight of greater than about 33 kDa. In another embodiment, the crosslinked network of entangled polypeptide chains is derived from a protein having greater than 300 residues. In an embodiment, the polypeptide chains have a length in an unfolded state of at least about 100 nm or at least about 200 nm. In another embodiment, the polypeptide chains have a length in an unfolded state of about 260 nm.

In an embodiment, the crosslinked network of entangled polypeptide chains is derived from an engineered protein. In another embodiment, the crosslinked network of entangled polypeptide chains is derived from a protein comprising ferredoxin-like folds.

In an embodiment, the crosslinked network of entangled polypeptide chains is derived from a protein comprising, consisting essentially of or consisting of (FL)ₓ, (FL-M23C)ₓ, (NuG2)ₓ, (GB1)ₓ, (GA)ₓ, where x is the number of protein repeat units and x is at least 4, GRG₅RG₄R, N₄RN₄RNR or combinations thereof. In another embodiment, the crosslinked network of entangled polypeptide chains is derived from a protein comprising (FL)ₓ, (FL-M23C)ₓ, (NuG2)ₓ, (GB1)ₓ, (GA)ₓ, where x is the number of protein repeat units and x is at least 4. In an embodiment, each x is independently an integer of from 4 to 20. In another embodiment, each x is independently an integer of from 4 to 16. In another embodiment, each x is independently an integer of from 4 to 12. In a further embodiment, x is 8. In another embodiment, x is 16. In another embodiment, the crosslinked network of entangled polypeptide chains is derived from a protein comprising, consisting essentially of or consisting of GRG₅RG₄R or N₄RN₄RNR. In an embodiment, the crosslinked network of entangled polypeptide chains is derived from a protein comprising, consisting essentially of or consisting of (FL)₈, (FL)₁₆, (FL-M23C)₈, (NuG2)₈, (GB1)₈ or (GA)₈. In another embodiment, the crosslinked network of entangled polypeptide chains is derived from a protein comprising, consisting essentially of or consisting of (FL)₈. In a further embodiment, the crosslinked network of entangled polypeptide chains is derived from a protein comprising (FL)₈. In another embodiment, the crosslinked network of entangled polypeptide chains is derived from a protein consisting essentially of (FL)₈. In another embodiment, the crosslinked network of entangled polypeptide chains is derived from a protein consisting of (FL)₈. In an embodiment, the crosslinked network of entangled polypeptide chains is derived from a protein comprising, consisting essentially of or consisting of (FL)₁₆. In another embodiment, the crosslinked network of entangled polypeptide chains is derived from a protein comprising, consisting essentially of or consisting of (FL-M23C)₈. In a further embodiment, the crosslinked network of entangled polypeptide chains is derived from a protein comprising, consisting essentially of or consisting of (NuG2)₈. In an embodiment, the crosslinked network of entangled polypeptide chains is derived from a protein comprising, consisting essentially of or consisting of (GB1)₈. In another embodiment, the crosslinked network of entangled polypeptide chains is derived from a protein comprising, consisting essentially of or consisting of (GA)₈.

The following are non-limiting examples of the present disclosure:

### EXAMPLES

### Example 1: Preparation of Tough and Stiff Protein Hydrogels

In order to engineer protein hydrogels with such a combination of mechanical features, higher crosslinking density and efficient microscopic mechanism for energy dissipation and recovery are desirable. Articular cartilage realized this unique combination of mechanical features by using a protein fiber-proteoglycan composite approach (Lu et al., 2008). However, this approach is difficult to design from bottom up (Xu et al., 2018; Yang et al., 2020). Conversely, muscles may provide an inspiration for an alternative approach for engineering cartilage-like protein-based materials. Forced-unfolding and refolding of globular proteins is a mechanism employed in muscle to allow for effective energy dissipation upon overstretching, and recovery upon relaxation (Linke et al., 1994; Rief et al., 1997; Li et al., 2002). This mechanism has been used to engineer protein hydrogels to mimic the passive elastic properties of muscles (Lv et al., 2010; Wu et al., 2018; Khoury et al., 2018; Fang et al., 2013). We reason that if an efficient method can be developed to significantly improve the stiffness of muscle-mimetic biomaterials, one should be able to take advantage of the forced-unfolding as an efficient energy dissipation mechanism to convert muscle-mimetic biomaterials into biomaterials with mechanical properties resembling those of articular cartilage.

Here we explore the feasibility to impart the soft, muscle-mimetic protein biomaterial with chain entanglement to significantly increase its stiffness. For this purpose, we used the elastomeric protein (FL)₈ as a model system.

By employing physical entanglements of protein chains and force-induced protein unfolding, here we report the engineering of a highly tough and stiff protein hydrogel to mimic articular cartilage. By crosslinking an engineered artificial elastomeric protein from its unfolded state, we introduced chain entanglement into the hydrogel network. Upon renaturation, the entangled protein chain network and forced protein unfolding entailed this single network protein hydrogel with superb mechanical properties in both tensile and compression tests, showing a Young's modulus of about 0.7 MPa and toughness of 250 kJ/m³ in tensile testing; and about 1.7 Mpa in compressive modulus and toughness of 3.2 MJ/m³. The energy dissipation in both tensile and compression tests is reversible and the hydrogel can recover its mechanical properties rapidly. Moreover, this hydrogel can withstand a compression stress of >60 Mpa without failure, amongst the highest compressive strength achieved by a hydrogel. These properties are comparable to those of articular cartilage, making this protein hydrogel useful, for example, as a novel cartilage-mimetic biomaterial. This study opened up a new potential avenue towards engineering protein hydrogel-based substitutes for articular cartilage, and may also help develop protein biomaterials with superb mechanical properties for other applications such as in soft actuators and/or robotics.

I. Methods

The (FL)₈ and (FL-M23C)₈ polyproteins were engineered using previously published protocols (Fang & Li, 2012). Protein hydrogels were constructed using a photochemical crosslinking strategy as described before. For the DC hydrogel, the photochemical crosslinking was carried out in 7 M guanidium hydrochloride (GdHCl) solution. For the NC hydrogel, the photochemical crosslinking was carried out in phosphate-buffered saline (PBS) solution. Tensile and compression tests were performed on an Instron-5500R universal testing system. The local slope at 15% strain on the loading curve was taken as the modulus for both tensile and compression tests. The toughness was calculated as the area between the loading curves at a given strain using custom-written software in IgorPro.

Protein engineering: FL domain is a redesigned variant of Di-I_5 (PDB code: 2KL8) (Koga et al.; Fang et al., 2013). The amino acid sequence of FL is: MGEFDIRFRT DDDEQFEKVL KEMNRRARKD AGTVTYTRDG NDFEIRITGI SEQNRKELAK EVERLAKEQN ITVTYTERGS LE. The genes of the polyprotein ferredoxin-like proteins (FL)₈, (FL-M23C)₈, and (FL)₁₆ were constructed following standard and well-established molecular biology methods as reported previously (Lv et al., 2010). Other polyproteins, (GB1)₈, (NuG2)₈, GRG₅RG₄R, N₄RN₄RNR and (GA)₈, were constructed following the same method. Polyprotein genes were inserted into the vector pQE80L for protein expression in E. coli strain DH5α. Seeding culture was allowed to grow overnight in 10 mL 2.5% Luria-Bertani broth (LB) medium containing 100 mg/L ampicillin. The overnight culture was used to inoculate 1 L of LB medium which was grown at 37° C. and 225 rpm for 3 hours to reach an OD₆₀₀ of about 0.8. Protein expression was induced with 1 mM isopropyl-1-β-D-thiogalactoside (IPTG) and continued at 37° C. for 4 hours. The cells were harvested by centrifugation at 4000 rpm for 10 mins at 4° C. and then frozen at −80° C. For polyprotein purification, cells were thawed and resuspended in 1× PBS and lysed by incubation with 1 mg/mL lysozyme for 30 mins. Nucleic acids were removed by adding 0.1 mg/mL of both Dnase and Rnase. The supernatant with soluble protein was collected after centrifuging the cell mixture at 12000 rpm for 60 mins. The soluble His6-tagged protein was purified using a Co²⁺ affinity column. The yields of (FL)₈, (FLM23C)₈, and (FL)₁₆ were approximately 80 mg, 80 mg and 45 mg respectively per liter of bacterial culture. Purified proteins were dialyzed extensively against deionized water for 2 days to remove residual NaCl, imidazole, and phosphate. Then the protein solution was filtered and lyophilized, and stored at room temperature until use. Bovine serum albumin (BSA) lyophilized powder was purchased from Sigma-Aldrich.

Hydrogel preparation and dimension measurements: Lyophilized native (FL)₈ protein was processed using two different gelation methods. The NC (native crosslinking) hydrogels (D-NC and N-NC) were prepared in native state by dissolving and gelating proteins in 1× PBS. After gelation, N-NC hydrogels were soaked in PBS, while the D-NC hydrogels were stored in 1× PBS containing 7M guanidine-hydrochloride (GdHCl) and achieved swelling equilibrium. The DC (denatured crosslinking) hydrogels (D-DC and N-DC) were prepared by dissolving the lyophilized (FL)₈ in 7M GdHCl for 2 hrs before use. The denatured protein solution was crosslinked into hydrogels and equilibrated in 7M GdHCl to obtain D-DC hydrogels, while N-DC hydrogels were renatured in PBS on a rocker by changing fresh PBS ten times over the course of 1 day until reaching equilibrium.

Gelation of (FL)₈, (FLM23C)₈ and (FL)₁₆ were based on a photochemical crosslinking strategy described previously (Fang et al., 2013; Lv et al., 2010; Fancy & Kodadek, 1999; Elvin et al., 2005). To prepare 20% (w/v) hydrogels, lyophilized proteins (200 mg/mL) were re-dissolved in PBS (D-NC and N-NC) or 7M GdHCl in PBS (D-DC and N-DC) respectively. A typical crosslinking reaction mixture contained 200 mg/mL of polyprotein, 50 mM ammonium persulfate (APmS) and 200 μM [Ru(bpy)₃]Cl₂. The protein mixture was quickly cast into a custom-made plexiglass ring-shaped mold (inner diameter, dᵢₙ=8 mm, outer diameter, dₒᵤₜ=10 mm, h=3 mm), and was exposed to a 200 W fiber optical white light source placed 10 cm above the mold for 10 min at room temperature. After gelation was complete, the hydrogel sample was carefully taken out of the mold. After the ring-shaped hydrogels were stored in the desired buffers for 3 hrs (N-DC gels for 24 hrs), the outer diameter, thickness, width and weight of all ring-shape equilibrated swollen/deswelling samples were measured before tensile tests. For compressive tests and SEM imaging, the hydrogels were prepared in a cylindrical shape following the same gelation procedures. The hydrogel preparation and the tensile (E) and compressive (Y) moduli measurements of (GB1)₈, (NuG2)₈, GRG₅RG₄R, NRN₄RN₄R, (GA)₈ and BSA followed the same procedures.

Swelling ratio and water content measurements: For swelling ratio and water content measurements, ring-shaped hydrogels were weighed immediately after being taken out of the mold, and the weight was recorded as mi. The swollen weight me was recorded after gently removing excess buffer from equilibrated hydrogels. To measure dry weight of the gels (m_{dry}), the hydrogels were firstly immersed in deionized water to remove extra salts, then lyophilized for 2 days and dried in a 70° C. incubator for 1 day. The swelling ratio (r) of the hydrogels was calculated using the following formula:

$$r = \frac{{m}_{s} - {m}_{i}}{{m}_{i}} \times 100 \% .$$

The water content (w) was determined by the dried gel (m_{dry}) and the equilibrated gel (me) (measured specimens, n=4):

$$w = ( 1 - \frac{{m}_{dry}}{{m}_{s}} ) \times 100 \% .$$

Tensile tests: Tensile tests were performed using an Instron-5500R tensometer with a custom-made force gauge and 5-N load transducer. The ring-shaped hydrogel specimen was stretched and relaxed in PBS (N-DC and N-NC) or 7 M GdHCl in PBS (D-DC and D-NC) at constant temperature (25° C.) without special preconditioning. The stress was calculated by dividing the load by the initial cross-sectional area of the hydrogel sample. The Young's modulus, breaking strain, and energy dissipation were measured using an extension rate of 25 mm/min. The stress at 15% strain is taken as the Young's modulus of the sample. Toughness was determined by integrating stress-strain curves where specimens were loaded directly to failure. Energy dissipation was calculated by integrating loop area between stretching and relaxing stress-strain curves. In hysteresis recovery experiments, a pulling rate of 200 mm/min was used. The same ring sample was stretched and relaxed with various time intervals.

Compression tests: Uniaxial compression tests were performed on cylinder-shaped hydrogels that were swollen to equilibrium using the Instron-5500R with 5000-N load transducer in air at room temperature. The dimensions (height (h₀) and diameter (d₀)) of the equilibrated N-DC and N-NC (FL)₈ hydrogel samples were: h₀: 3.0 mm and d₀: 6.5 mm for the N-DC hydrogel, and h₀: 5.0 mm and d₀: 5.0 mm for the N-NC hydrogel. The gel was put on the lower plate, while the upper plate approached the sample slowly until a rise in force was detected, indicating contact between the plate and the gel. The stress was calculated by dividing the load by the initial cross-sectional area of the hydrogel sample. Unless a different rate is stated, the gel was compressed and relaxed at a compression speed of 2 mm/min. No water was squeezed out of the gels during compression. The compressive modulus was measured at a strain of 10-20%. The maximum stress and strain were determined at failure points, where the first crack in the gel was observed. Energy dissipation was calculated by integrating loop area between compressing and relaxing stress-strain curves (n=7). In hysteresis recovery experiments, a compression rate of 100 mm/min was used.

Scanning electron microscopy (SEM) imaging: 20% (w/v) D-NC and N-NC (FL)₈ hydrogel samples were prepared for SEM imaging using a Hitachi 54700 scanning electron microscope. The samples were then shock-frozen in liquid nitrogen, and quickly transferred to a freeze drier where they were lyophilized for 24 hrs. Lyophilized samples were then carefully fractured in liquid nitrogen, and fixed on aluminum stubs. The sample surface was coated by 8 nm of gold prior to SEM measurements.

Characterization of dityrosine cross-links in hydrogels by acid hydrolysis-fluorescence method: The degree of dityrosine crosslinking in both NC and DC (FL)₈ hydrogels was characterized following a well-established fluorimetry method (Fang & Li, 2012). Dityrosine emits at a wavelength of 410 nm when excited at 315 nm. For quantification of the dityrosine and dityrosine-like compounds generated in NC and DC (FL)₈ hydrogels, we followed a well-established fluorescence standard curve method (Fang & Li, 2012). Typically, 20% (w/v) hydrogel samples (about 25 mg) were reacted with 100 μL HCl (6 N) in a sealed 1.5 mL centrifuge tube in a metal heat block at 105° C. for 2 hrs to achieve full hydrolysis of the peptide bonds. Then, 100 μL of acid hydrolysis product was transferred into anew 1.5 mL centrifuge tube and neutralized by 10 μL NaOH (5 M). Next, 100 mM Na₂CO₃-NaHCO₃ buffer (pH 9.9) was added to a final volume of 1 mL. Fluorescence spectra of the samples were measured by a Varian Cary Eclipse fluorescence spectrophotometer. According to the fluorescence-concentration standard calibration curve of dityrosine, the yield of dityrosine and dityrosine-like products in the hydrogel was then determined (n=8).

Cysteine shotgun fluorescence labeling by IAEDANS and fluorescence measurements: DC and NC (FLM23C)₈ hydrogels for cysteine shotgun labeling were prepared with the same protein concentration and gel preparation procedures as the wild-type (FL)₈. The labeling reaction was performed in the dark at room temperature for 3 hrs in PBS buffer (pH 7.4) containing 5 mM TCEP and 2 mM 5-((2-[iodoacetyl)amino]ethyl)amino)naphthalene-1-sulfonic acid (IAEDANS). As a control, D-DC and D-NC (FLM23C)₈ hydrogels incubated in 7 M GdHCl (containing 5 mM TCEP, pH 7.4) were also labeled using IAEDANS. Then all hydrogels were transferred to PBS buffer containing 5 mM β-mercaptoethanol to quench the reaction. To remove excess labeling dye, additional PBS buffer (containing 5 mM β-mercaptoethanol) was added, and changed five times over the course of 5 hrs until fluorescence could no longer be detected in the buffer solution. To quantify the fluorescence intensity of IAEDANS labeled hydrogels, the hydrogels were digested with trypsin at 37° C. for 5 hrs. The digestion reaction contained 5% trypsin (relative to the hydrogel weight), 25 mM NH₄HCO₃, mM CaCl₂), 1 M GdHCl and 10 mM dithiothreitol. Unlabeled (FLM23C)₈ hydrogel was digested in the same way to serve as a negative control. After digestion, 50 μL of the digested mixture was diluted to 3 mL using PBS buffer. The fluorescence emission spectrum was measured by a Varian Cary Eclipse fluorescence spectrometer using an excitation wavelength of 336 nm and emission at 490 nm was monitored. An IAEDANS standard calibration curve was also created, covering linear concentration range of 0-60 μM (n=8).

Single-molecule optical tweezers measurements: Single-molecule optical tweezer measurements were carried out using a MiniTweezers setup (http://tweezerslab.unipr.it) as previously described (Lei et al., 2017). Sample preparation including the protein—DNA construct formation and force measurement protocols was adapted from protocols described previously (Lei et al., 2017). Force-distance curves of the protein—DNA construct were obtained using constant velocity pulling protocol.

II. Results and Discussion

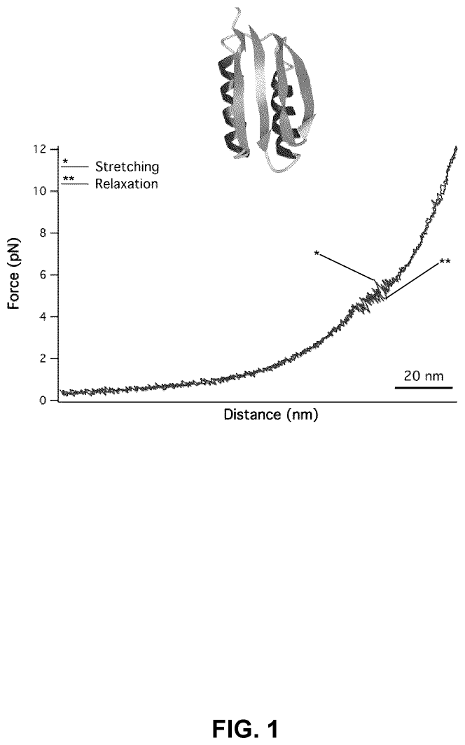

FL is a de novo designed ferredoxin-like globular protein (Koga et al., 2012). Single molecule optical tweezers experiments showed that FL is mechanically labile, and unfolds and refolds readily at about 5 pN (FIG. 1; Fan et al, 2013). The unfolding-refolding of FL occurred at about 5 pN, making FL a mechanically labile protein. The elastomeric protein (FL)₈ was used to engineer highly stretchy and tough protein hydrogels, in which the forced-unfolding of FL domains served as a highly effective means in dissipating energy in the hydrogel (Fang et al., 2013). However, the Young's modulus of the (FL)₈ hydrogel is only about 15 kPa (Fang et al., 2013). We sought to use (FL)₈ as a model system for enhancing its mechanical stiffness.

The molecular weight of (FL)₈ is about 80 kDa, but its contour length in its native state is only about 10 nm. Thus, there is no chain entanglement in the native (FL)₈ hydrogels. However, in its unfolded state, (FL)₈ is about 260 nm long, showing the characteristic length of a polymer chain. In the concentrated solution of unfolded (FL)₈ (>150 mg/mL), the unfolded polypeptide chains will overlap and likely entangle (Colby, 2010). We reasoned that if unfolded (FL)₈ is crosslinked from its concentrated solution, inter-chain entanglement could be trapped by the chemical crosslinks in the crosslinked hydrogel network.

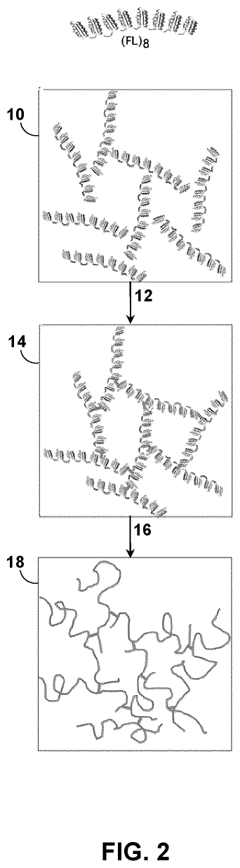

To test this feasibility, the well-developed [Ru(bpy)₃]²⁺-mediated photochemical crosslinking strategy, which crosslinks two tyrosine residues in proximity into a dityrosine adduct (Lv et al., 2010; Elvin et al., 2005; Fancy & Kodadek, 1999; Partlow et al., 2016), was employed to construct (FL)₈ hydrogels. We used the DC (denatured crosslinking) method to construct the denatured (FL)₈ hydrogels, in which the unfolded (FL)₈ was photochemically crosslinked into hydrogels directly from its concentrated solution (20%, 200 mg/mL) in 7 M guanidine hydrochloride (GdHCl). The as-prepared hydrogel was then equilibrated in 7 M GdHCl to obtain the denatured DC hydrogel (referred to herein as the D-DC hydrogel). As a control, we also constructed a denatured (FL)₈ hydrogel that is free of chain entanglement using the NC (native crosslinking) method (FIG. 2). The native elastomeric protein (FL)₈ (top) was first dissolved in PBS to a high concentration (about 200 mg/mL) to form native protein solution 10. Upon photochemical crosslinking 12, (FL)₈ was crosslinked into a hydrogel network without chain entanglements, due to the short length of folded (FL)₈, resulting in the N-NC hydrogel 14. Then the prepared hydrogel was denatured 16 and equilibrated in 7M GdHCl to obtain the denatured NC hydrogel (referred to as the D-NC hydrogel). When denatured 16 in GdHCl, the (FL)₈ in the hydrogel network unfolded and behaved as random coils. The resultant D-NC hydrogel 18 is also free of chain entanglement.

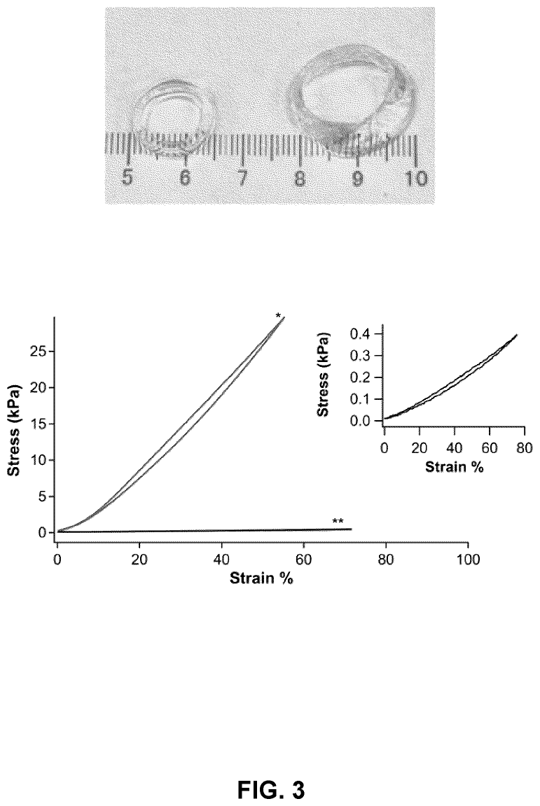

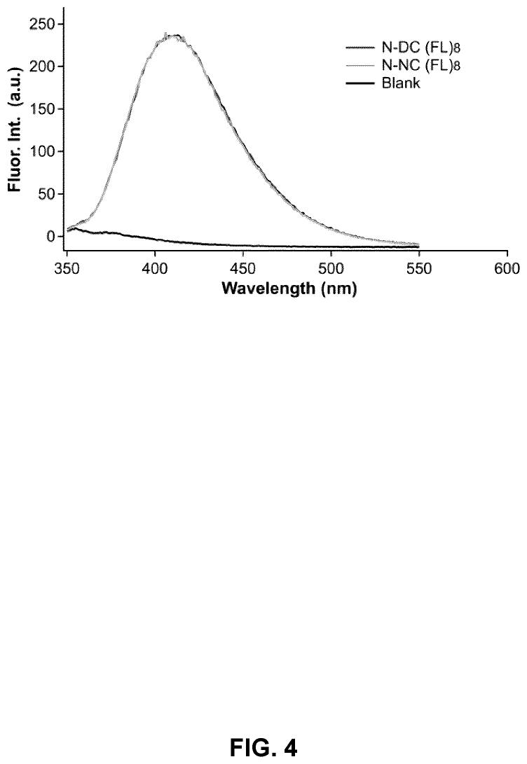

FIG. 3 (top) shows the photographs of both D-DC (left) and D-NC (right) (FL)₈ hydrogels prepared using the same ring-shaped mold as well as their stress-strain curves (bottom). Evidently, the D-DC hydrogel was self-standing and swelled to a much less degree than the D-NC hydrogel, while the D-NC hydrogel ring-shaped sample collapsed onto itself. The D-DC hydrogel displayed a Young's modulus of 56 kPa, significantly higher than that of D-NC hydrogel (about 1 kPa). According to the classical rubber elasticity theory (Treloar, 1975), G=ρRT/M_{c}, where G is the shear modulus, ρ is the density of the polymer in the network, Mc is the average molecular weight between two neighboring crosslinking points, R is the gas constant and T is temperature, the stiffness (Young's modulus) of a hydrogel network is determined by the crosslinking density. Thus, our results suggested a much higher effective crosslinking density for the D-DC gel than the D-NC gel. We quantified the number of dityrosine adducts by measuring the characteristic dityrosine fluorescence (Elvin et al., 2005; Fang & Li, 2012) to examine if these two preparation methods led to different number of dityrosine crosslinking points in the (FL)₈ hydrogel, (FIG. 4). Our results showed that both hydrogels contained roughly the same number of dityrosine crosslinking points (about 17% of the total number of tyrosine residues in FL domains were cross-linked into dityrosine adducts). Since the D-DC and D-NC hydrogels had the same number of crosslinking points, the same unfolded protein chain and the same protein chain-solvent interaction, the higher effective crosslinking density of the D-DC hydrogel should originate from additional effective crosslinking points resulting from the chain entanglements of unfolded (FL)₈ polypeptide chains in the D-DC hydrogel network. Evidently, the chain entanglement significantly enhanced the stiffness of the denatured (FL)₈ hydrogels.

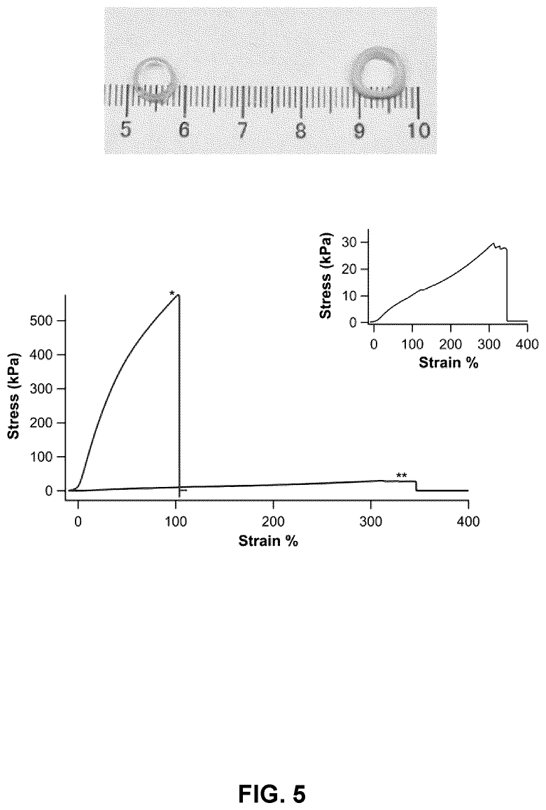

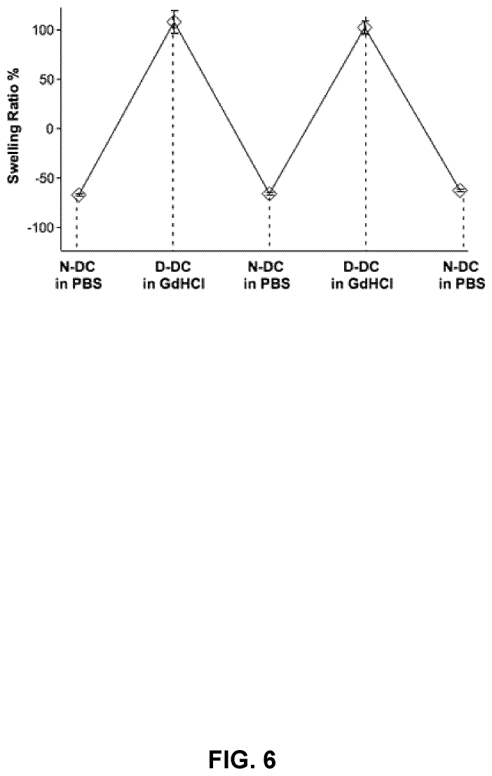

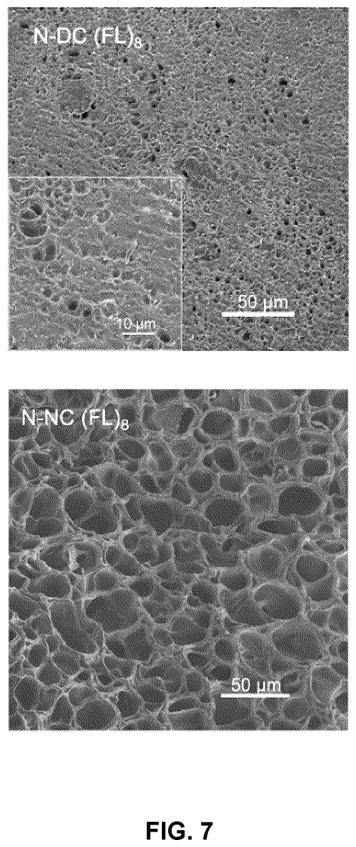

Chain entanglement in a chemically crosslinked hydrogel network cannot be removed without disrupting the chemical crosslinking network. Thus, chain entanglement will be retained in the hydrogel network even if the polypeptide chains undergo conformational changes, such as folding. Taking advantage of this unique topological feature, we “renatured” the D-DC hydrogel in PBS buffer to allow the refolding of FL domain to obtain the native DC (N-DC) (FL)₈ hydrogel. We observed that after renaturing in PBS, the N-DC hydrogel deswelled dramatically compared with the D-DC hydrogel, and changed from being transparent to largely translucent (FIG. 5). In contrast, upon renaturation, the N-NC hydrogel deswelled and became opaque. The swelling ratio of the N-DC hydrogel was smaller than that of the N-NC hydrogel. Moreover, both hydrogels can be cycled between their native and denatured states (N-DC to D-DC, N-NC to D-NC) for many cycles without noticeable change in their respective physical appearances and properties (FIG. 6). While not wishing to be limited by theory, the deswelling is likely due to the refolding of some FL domains in PBS, which is accompanied by a significant shortening of the contour length of the polyprotein (from 260 nm to 10 nm). Microscopically, both N-DC and N-NC hydrogels showed microporous structures, but the mesh size of N-DC hydrogel was significantly smaller than that of the N-NC hydrogel, consistent with the smaller swelling ratio of the N-DC hydrogel (FIG. 7).

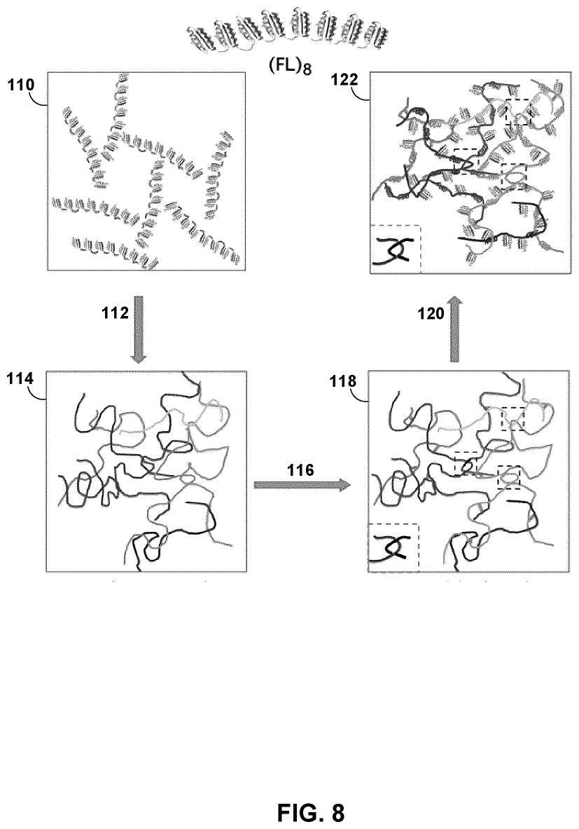

During renaturation, due to the presence of chain entanglement and the shortening of the polypeptide chain, it is expected that some FL domains would not be able to fold into their native state. Instead, while not wishing to be limited by theory, they likely assumed a hydrophobically collapsed state and/or random coil state, as PBS is a poor solvent for the unfolded FL domain. Therefore, while not wishing to be limited by theory, it is likely that the N-DC (FL)₈ hydrogel network assumed a single network structure made up of folded FL domains as well as unfolded ones, as schematically shown in FIG. 8. Referring to the schematics of the preparation of D-DC and N-DC (FL)₈ hydrogels in FIG. 8, the elastomeric protein (FL)₈ was first dissolved in PBS to high concentration (about 200 mg/mL) to obtain the native protein solution 110. When denatured 112 in GdHCl, the unfolded (FL)₈ polypeptide chains in the denatured protein solution 114 behaved as random coils, which overlapped with one another due to high protein concentration, leading to possible chain entanglements. Upon photochemical crosslinking 116, chain entanglements were retained or created, leading to a network of entangled polypeptide chains, in the D-DC hydrogel 118. Entangled chains are highlighted in dashed squares. The lower left bottom shows a zoomed view of one such chain entanglement. When the D-DC hydrogel was equilibrated in PBS (renaturation 120), some FL domains refolded, while others underwent hydrophobic collapse. In the renatured hydrogel (N-DC hydrogel 122), the chain entanglements remained, which are highlighted in dashed squares. For clarity, different molecules in 114, 118 and 122 are shaded differently.

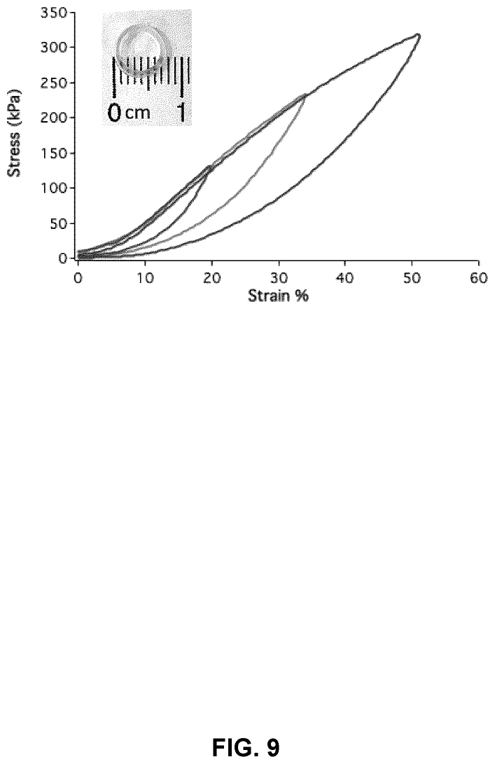

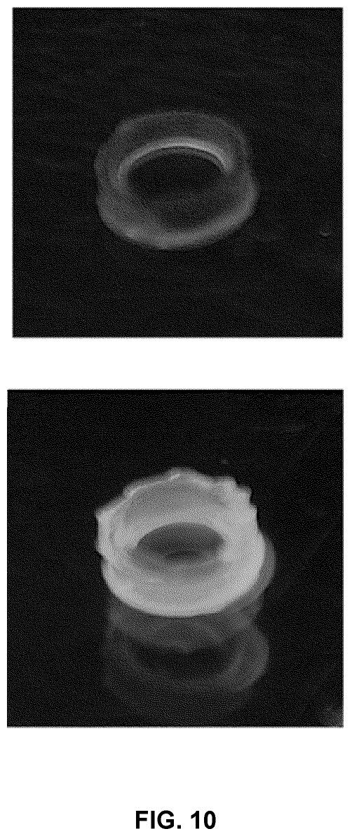

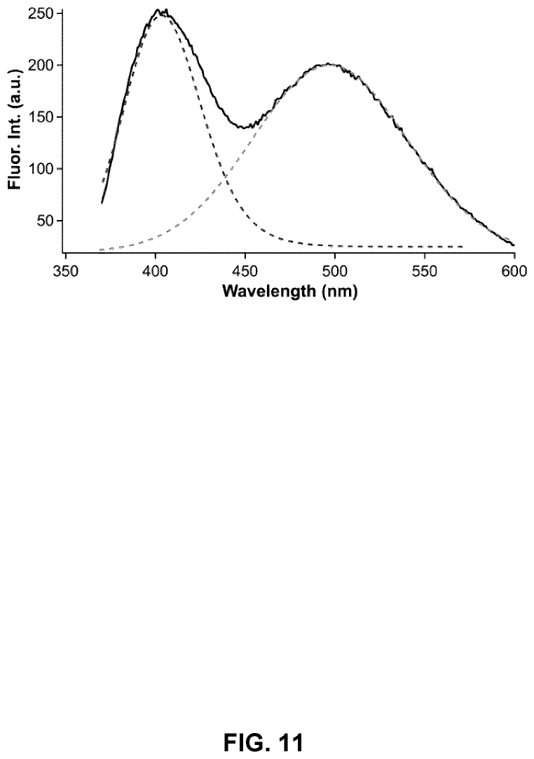

To verify the existence of unfolded FL domains in the N-DC hydrogel, we used a well-established cysteine shotgun labeling approach, which allows for labeling of only solvent-exposed cysteine residues using the thiol reactive fluorescent dye 5-((2-((iodoacetyl)amino)ethyl)amino)naphthalene-1-sulfonic acid (IAEDANS) (Johnson et al., 2007). For this, we used a cysteine variant FL-M23C, where the buried residue Met23 in FL was mutated to Cys. Cys23 is sequestered in the hydrophobic core of the folded FL and can only be labeled with IAEDANS when FL-M23C is unfolded (Fang & Li, 2012). The (FLM23C)₈-based hydrogels showed similar physical and mechanical properties as (FL)₈ (FIG. 9). The mechanical properties of (FM23C)₈ hydrogels are similar to those of (FL)₈ hydrogels. The Young's modulus was 0.89 MPa. After IAEDANS labeling in PBS, the N-DC hydrogel showed the characteristic cyan fluorescence of IAEDANS under UV illumination (FIG. 10 and FIG. 11). Quantitative analysis showed that about 50% of the FL domains were unfolded in the N-DC hydrogel. In contrast, only about 23% of the FL domains were unfolded in the N-NC hydrogel (Fang & Li, 2012). These results indicated that the N-DC hydrogel is indeed a single network hydrogel consisting of folded and unfolded FL domains.

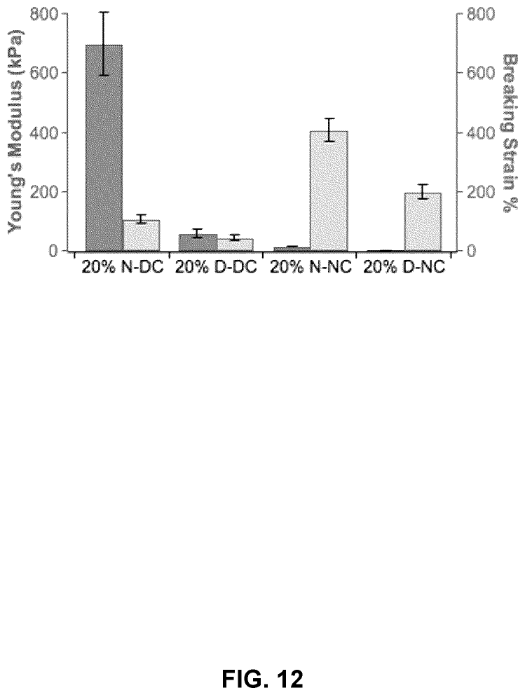

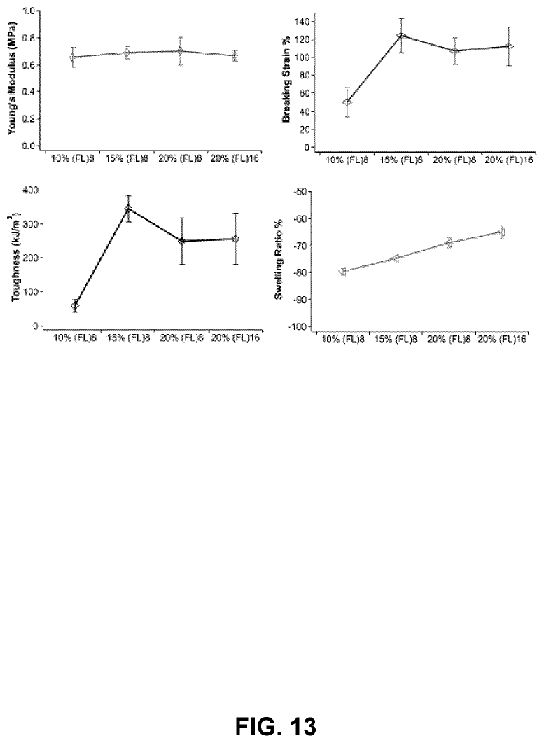

We then carried out tensile and compression tests to measure the mechanical properties of the N-DC (FL)₈ hydrogel. A typical stress-strain curve of the N-DC hydrogel in PBS buffer is shown in FIG. 5, where the N-DC hydrogel was stretched to failure. For comparison, the stress-strain curve of the N-NC hydrogel is also shown. It is evident that the stiffness of the N-DC hydrogel increased dramatically compared with that of the N-NC hydrogel and D-DC hydrogel. The N-DC hydrogel displayed a Young's modulus of 0.70±MPa (n=18) (FIG. 5 and FIG. 12), significantly higher than that of protein-based hydrogels reported in the literature (Lv et al., 2010; Wu et al., 2018; Elvin et al., 2005; McGann et al., 2013; Partlow et al., 2014). The breaking strain of the N-DC hydrogel was 107±14%, indicative of its good stretchability (FIG. 12). Similar Young's modulus and breaking strain were also observed for 15% N-DC (FL)₈ hydrogel as well as N-DC hydrogels constructed from (FL)₁₆. However, 10% N-DC (FL)₈ hydrogel showed similar Young's modulus but much smaller breaking strain (50%) (FIG. 13).

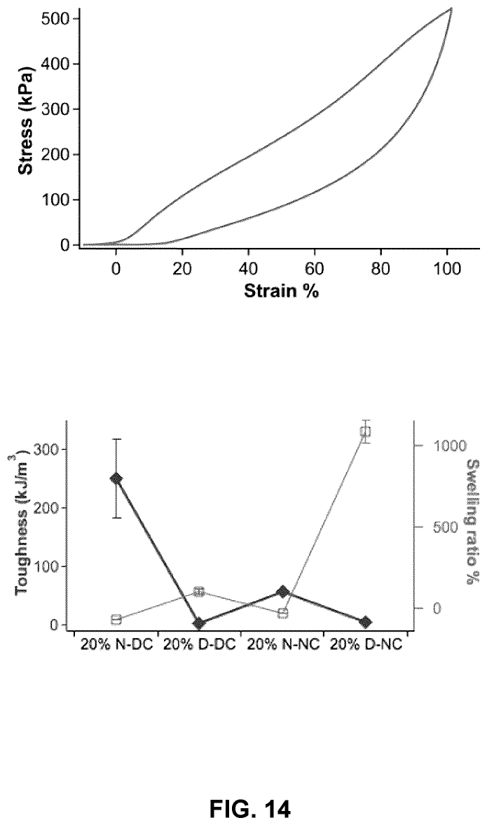

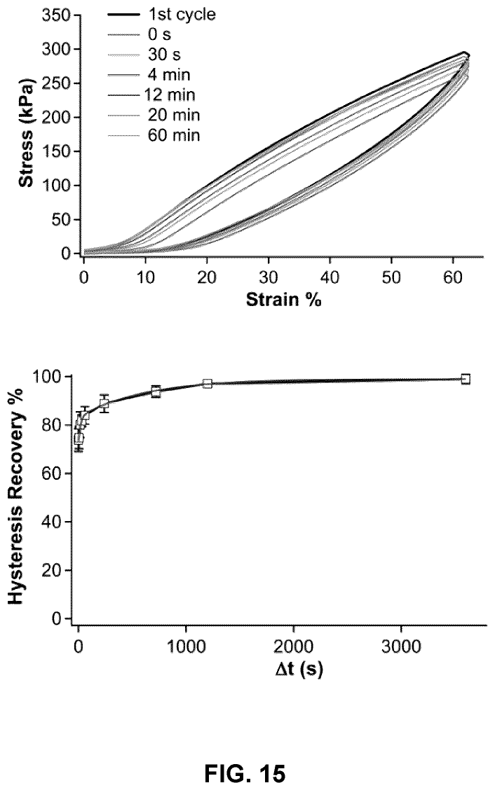

The N-DC (FL)₈ hydrogels can dissipate a large amount of energy. For example, during cyclic stretching-relaxation experiments, the N-DC hydrogel exhibited a large hysteresis, indicative of a significant amount of energy dissipation at high strains during stretching (FIG. 14, top). The average energy dissipation of the N-DC hydrogel is 250±68 kJ/m³, demonstrating its superb toughness (FIG. 14, bottom). Moreover, the energy dissipation of the N-DC (FL)₈ hydrogel was fully reversible, and the hysteresis can be recovered rapidly once the hydrogel was relaxed to zero strain (FIG. 15, top). After being stretched to 60% strain and then relaxed to zero strain, 70% of the hysteresis was recovered right after the hydrogel was relaxed, and the rest 30% hysteresis recovered more slowly following a double-exponential kinetics, with a rate constant k₁ of 0.05±0.02 s⁻¹ and k₂ of (1.7±0.3)×10⁻³ s⁻¹, respectively (FIG. 15, bottom). While not wishing to be limited by theory, this reversible hysteresis is likely due to the forced-unfolding, and subsequent refolding of the FL domains in the hydrogel network, consistent with the fast refolding rate of FL domain at zero force (Fang et al., 2013).

Evidently, the N-DC (FL)₈ hydrogels displayed tensile mechanical properties that uniquely combined a high Young's modulus (about 0.7 MPa), high toughness as well as fast recovery, a combination that is difficult to achieve as individual properties are often mutually incompatible. Additionally, the high Young's modulus and toughness of this single network protein hydrogel are amongst the highest of the engineered protein hydrogels, and even compare favorably with those of some specially designed synthetic polymer hydrogels of special network structures, such as double network hydrogels (Gong et al., 2003; Gong, 2010), or polymer composite hydrogels (Xu et al., 2018).

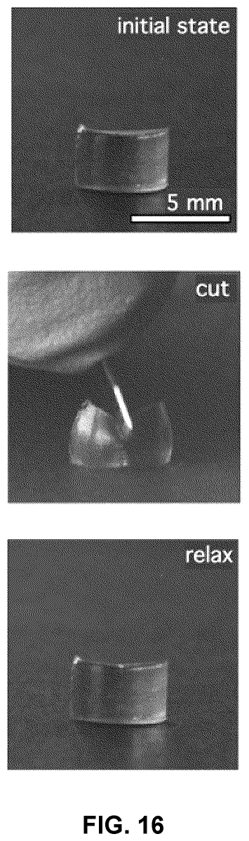

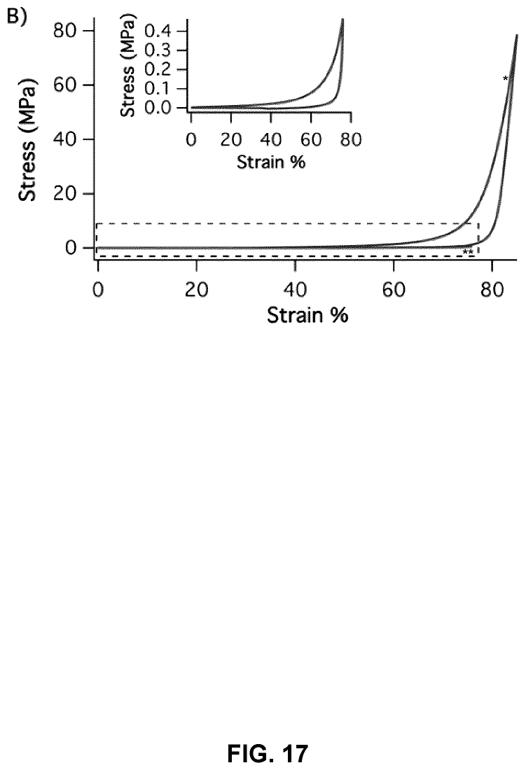

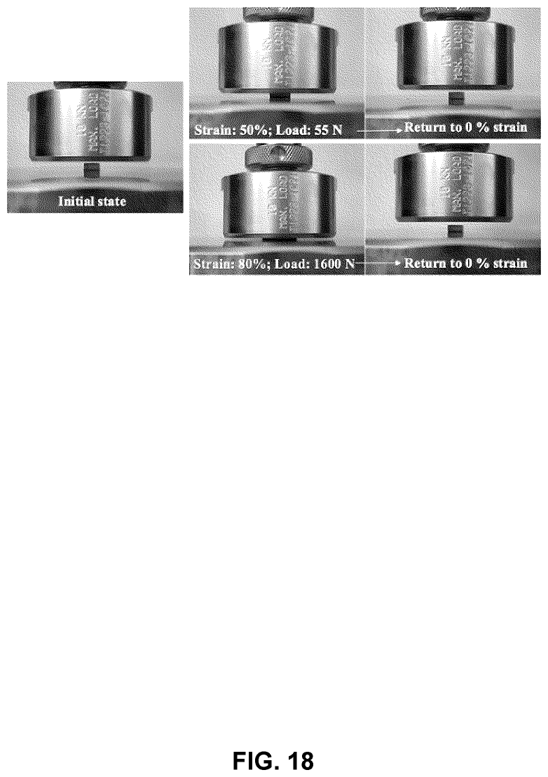

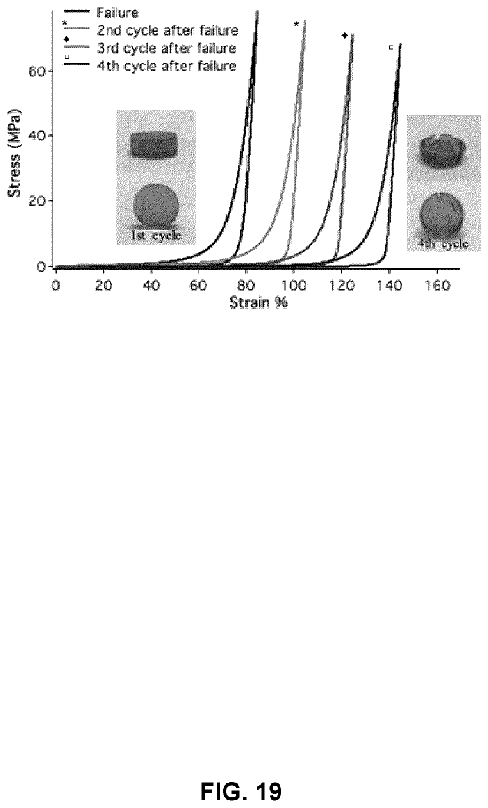

In addition to these superb tensile properties, the N-DC (FL)₈ hydrogel demonstrated even more striking compressive mechanical properties. We found that the N-DC (FL)₈ hydrogel is super tough and can resist slicing with a sharp scalpel, despite that it contains about 60% water (FIG. 16). To quantify the compressive mechanical properties of the N-DC (FL)₈ hydrogel, standard compression tests were carried out (FIG. 17). The stress-strain curves showed that the N-DC (FL)₈ hydrogels displayed a compressive modulus of about 1.7 MPa at strain. In comparison, the N-NC (FL)₈ hydrogels only showed a compressive modulus of about 50 kPa (FIG. 17, inset), again revealing the significant enhancement effect of chain entanglement on the stiffness of the (FL)₈ hydrogels. As the strain increased, the stress of the N-DC (FL)₈ hydrogel showed a much more pronounced increase: the modulus at 75% strain reached about 10 MPa. Strikingly, the N-DC FL hydrogel could be compressed to more than 80% strain and sustain a compressive stress of as high as 75 MPa without fracture (FIG. 18). The average compressive strength of the N-DC (FL)₈ hydrogel was 68±12 MPa (n=7), and an average failure strain of 82±3%. At the failure strain, a small crack often started to appear in the hydrogel sample. However, the failure was not brittle, as evidenced by the subsequent stress-strain curves of the same hydrogel sample (FIG. 19). These results demonstrated the super high compressive strength of the N-DC (FL)₈ hydrogel. The compressive strength of the N-DC (FL)₈ hydrogel is amongst the highest strength achieved by hydrogels (Table 1), and compares favorably with that of articular cartilage (Hayes & Mockros, 1971; Kerin et al., 1998; Lu & Mow, 2008). As a comparison, the super tough double network polymer hydrogels typically fractured at a stress of no more than 20 MPa (Gong et al., 2003; Gong, 2010).

TABLE 1
Mechanical properties of protein hydrogels and polymer hydrogels

Tensile

| | | | Young's | | Breaking | | Breaking | |
|---|---|---|---|---|---|---|---|---|
| | | | modulus | | stress | | strain | Reference(s) |
| Cartilage | Articular | | 1-10 | MPa | 1-20 | MPa | — | Kerin et al., |
| | cartilage | | | | | | | 1998; |
| | | | | | | | | Williamson et |
| | | | | | | | | al., 2003; |
| | | | | | | | | Wainwright et |
| | | | | | | | | al., 1982. |
| Current | (FL)₈ | | 0.7 | MPa | 0.6 | MPa | 107% | — |
| work | | | | | | | | |
| Protein | GRG5RG4R | | 50 | kPa | 70 | kPa | 100% | Lv et al., |
| Hydrogels | | | | | | | | 2010 |
| | (FL)₈ | | 16 | kPa | 35 | kPa | 450% | Fang et al., |
| | | | | | | | | 2013 |
| | Bovine fibrinogen | | 70 | kPa | 46 | kPa | 62% | Elvin et al., |
| | | | | | | | | 2009 |
| | Gelatin | | 70 | kPa | — | | — | Gan et al., |
| | | | | | | | | 2019 |
| | Gelatin | | 67 | kPa | 91 | kPa | 145% | Elvin et al., |
| | | | | | | | | 2010 |
| | Regenerated silk | | 50 | kPa | — | | — | Partlow et al., |
| | fibroin | | | | | | | 2014 |
| | Gell crosslinked with | | 10-150 | kPa | 20 | kPa | 16% | Wu et al., |
| | 4-arm PEG | | | | | | | 2018 |
| | (GB1)8-RX3 | | 65 | kPa | 110 | kPa | 80% | Fang & Li, |
| | | | | | | | | 2012 |
| | Regenerated silk | | 2 | MPa | 0.7 | MPa | 110% | Su et al., |
| | fibroin (alcohol | | | | | | | 2017 |
| | treated) | | | | | | | |
| | Regenerated silk | | — | | — | | — | Numata et al., |
| | fibroin | | | | | | | 2017 |
| | (alcohol treated | | | | | | | |
| | double network) | | | | | | | |
| Polymer | Double | PAMPS- | — | | — | | — | Gong et al., |
| hydrogels | network | PMAAm | | | | | | 2003 |
| | | Bacterial | — | | — | | — | Nakayama et |
| | | cellulose- | | | | | | al., 2004 |
| | | PDMAAm | | | | | | |
| | | PAMPS- | — | | — | | — | Gong et al., |
| | | collagen | | | | | | 2003 |
| | Cojoined | Chitosan- | 2.5 | MPa | 4.3 | MPa | 350% | Xu et al., |
| | network | gelatin- | | | | | | 2019 |
| | | phytate | | | | | | |
| | Slide- | NIPA- | 43 | kPa | 40 | kPa | 450% | Bin Imran et |
| | ring | AAcNa- | | | | | | al., 2014 |
| | network | HPR-C | | | | | | |

Compression

| | | | Compressive | | Breaking | | Breaking | |
|---|---|---|---|---|---|---|---|---|
| | | | modulus | | stress | | strain | Reference(s) |
| Cartilage | Articular | | 0.5-10 | MPa | 10-50 | MPa | — | Kerin et al., |
| | cartilage | | | | | | | 1998; |
| | | | | | | | | Williamson et |
| | | | | | | | | al., 2003; |
| | | | | | | | | Wainwright et |
| | | | | | | | | al., 1982. |
| Current | (FL)₈ | | 1.7 | MPa | 68 | MPa | 82% | — |
| work | | | | | | | | |
| Protein | GRG5RG4R | | — | | — | | — | — |
| Hydrogels | (FL)₈ | | — | | — | | — | Fang et al., |
| | | | | | | | | 2013 |
| | Bovine fibrinogen | | — | | — | | — | Elvin et al., |
| | | | | | | | | 2009 |
| | Gelatin | | 175 | kPa | 0.5 | MPa | — | Gan et al., |
| | | | | | | | | 2019 |
| | Gelatin | | — | | — | | — | Elvin et al., |
| | | | | | | | | 2010 |
| | Regenerated silk | | 20 | kPa | — | | — | Partlow et al., |
| | fibroin | | | | | | | 2014 |
| | Gell crosslinked with | | — | | — | | — | Wu et al., |
| | 4-arm PEG | | | | | | | 2018 |
| | (GB1)8-RX3 | | — | | — | | — | Fang & Li, |
| | | | | | | | | 2012 |
| | Regenerated silk | | 0.3-3 | MPa | 0.5-1.7 | MPa | about 60% | Su et al., |
| | fibroin (alcohol | | | | | | | 2017 |
| | treated) | | | | | | | |
| | Regenerated silk | | 5.8 | MPa | 12.4 | MPa | 80% | Numata et al., |
| | fibroin | | | | | | | 2017 |
| | (alcohol treated | | | | | | | |
| | double network) | | | | | | | |
| Polymer | Double | PAMPS- | 0.33 | MPa | 17 | MPa | 92% | Gong et al., |
| hydrogels | network | PMAAm | | | | | | 2003 |
| | | Bacterial | 1.6 | MPa | 2.9 | MPa | 50% | Nakayama et |
| | | cellulose- | | | | | | al., 2004 |
| | | PDMAAm | | | | | | |
| | | PAMPS- | — | | 2.9 | MPa | 53% | Gong et al., |
| | | collagen | | | | | | 2003 |
| | Cojoined | Chitosan- | 6.6 | MPa | 64 | MPa | 87% | Xu et al., |
| | network | gelatin- | | | | | | 2019 |
| | | phytate | | | | | | |
| | Slide- | NIPA- | — | | — | | — | Bin Imran et |
| | ring | AAcNa- | | | | | | al., 2014 |
| | network | HPR-C | | | | | | |

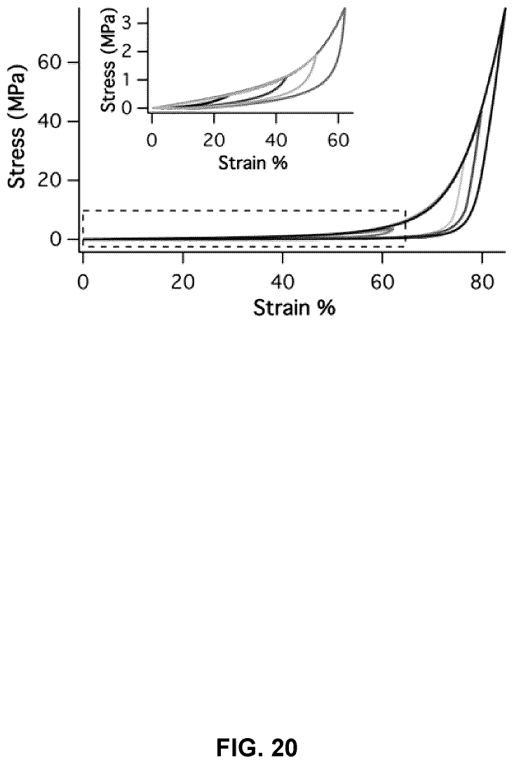

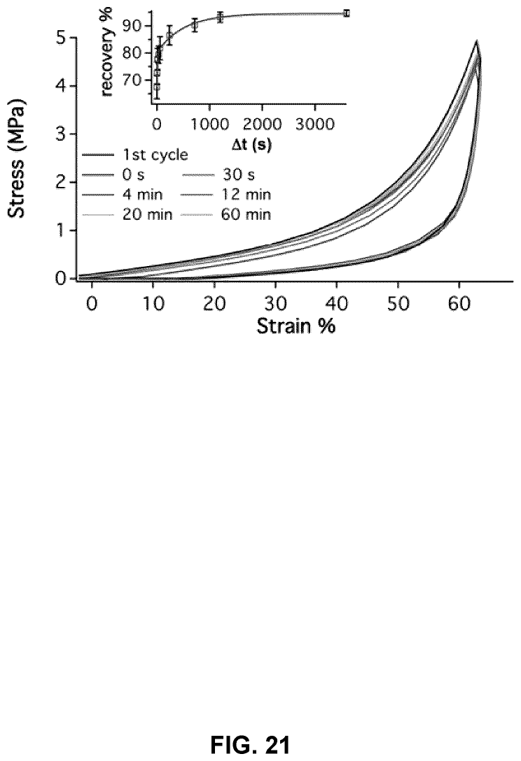

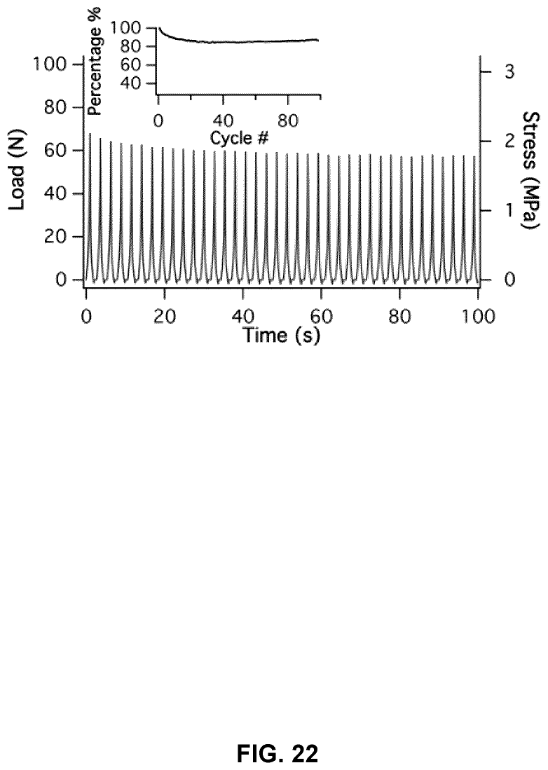

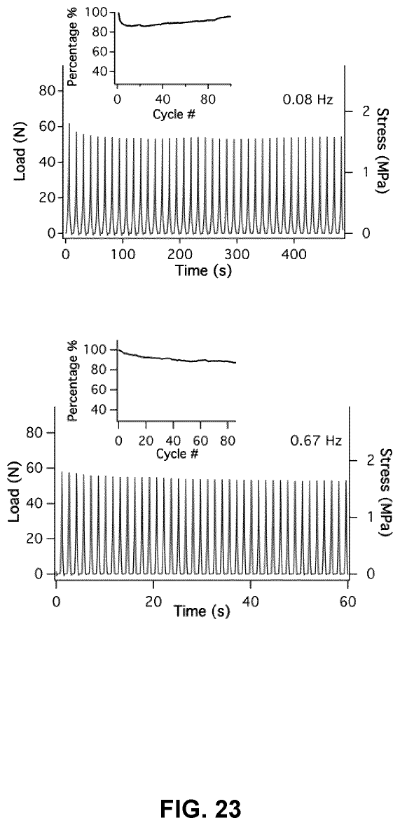

In the compression-unloading cycles, a large hysteresis was observed (FIG. 20), indicative of a large amount of energy being dissipated during compression. The hysteresis increased as the strain increased. At 80% strain, the hydrogel exhibited a toughness of 3.2±0.6 MJ/m³ (n=7). Moreover, the (FL)₈ hydrogel displayed superb recovery properties (FIG. 21). At lower strains (<40%), the hydrogel recovered its dimension right at the end of the loading-unloading cycles, suggesting that the recovery of the hydrogel is fast. Even at a large compression strain (>60%), about 65% of the hysteresis can be recovered right after unloading. The remaining 35% hysteresis could be recovered within an hour following a double exponential kinetics (FIG. 21). Furthermore, after 100 consecutive loading-unloading cycles at a frequency of 0.37 Hz and a final strain of 50%, the consecutive loading-unloading curves did not show much change, and the stress of the N-DC (FL)₈ hydrogel at 50% strain retained about 90% of its original stress (FIG. 22). Similar results were obtained at a frequency of 0.67 Hz and 0.08 Hz (FIG. 23), demonstrating that the N-DC (FL)₈ hydrogel can quickly recover its mechanical properties and do not show much fatigue.

Collectively, these results revealed that the N-DC (FL)₈ hydrogels are mechanically strong and tough, and can recover their shape and mechanical properties rapidly and do not show much mechanical fatigue. Moreover, these protein hydrogels showed excellent long-term stability: after being stored in PBS (with 0.2‰NaN₃) for over six months, their physical shape and mechanical properties remained largely unchanged. These exceptional mechanical properties and their unique integration in one material are rare for protein hydrogels, and compare favorably with those of polymer hydrogels with special network structure (Table 1). These properties closely reproduced many features of articular cartilage, and thus make the N-DC (FL)₈ hydrogels for example, a cartilage-mimetic protein biomaterial.

While not wishing to be limited by theory, these superb mechanical properties of N-DC (FL)₈ hydrogels are likely from a combination of factors, including chain entanglement, folded and hydrophobically collapsed FL domains in the hydrogel network, as well as the forced unfolding and refolding of FL domains. These unique features likely make this DC method for protein hydrogelation broadly applicable. Indeed, as shown in Table 2, in protein hydrogels constructed from a range of elastomeric proteins, which range from all α proteins to α/β proteins, we significantly enhanced their stiffness via this DC hydrogelation method and improved their Young's modulus to several hundred kPa, depending on the inherent tyrosine content of the elastomeric protein. Similar enhancement was also achieved in the compressive modulus of these protein hydrogels (Table 2). These results have demonstrated the generality of this new method.

TABLE 2
Enhancement of mechanical properties via the DC hydrogelation method

| | Tensile | | | Compression | | |
|---|---|---|---|---|---|---|
| | N-DC | N-NC | | N-DC | N-NC | |
| | hydrogel | hydrogel | | hydrogel | hydrogel | |
| | E_{N-DC} | E_{N-NC} | Enhancement | Y_{N-DC} | Y_{N-NC} | Enhancement |
| Protein | (kPa) | (kPa) | E_{N-DC}/E_{N-NC} | (kPa) | (kPa) | Y_{N-DC}/Y_{N-NC} |
| (FL)₈ | 700 ± 110 | 15.7 ± 1.8 | 44 | 1614 ± 201 | 40.1 ± 2.7 | 40 |
| (NuG2)₈ | 620 ± 93 | 11.2 ± 3.3 | 56 | 1086 ± 31 | 55.0 ± 11 | 20 |
| (GB1)₈ | 227 ± 30 | 4.8 ± 1.1 | 47 | 339 ± 36 | 68.3 ± 10.5 | 5.0 |
| GRG₅RG₄R | 123 ± 53 | 32.7 ± 4.2 | 3.8 | 220 ± 31 | 19.6 ± 3.9 | 11 |
| N₄RN₄RNR | 126 ± 21 | 22.1 ± 2.7 | 5.7 | 592 ± 148 | 114.0 ± 25.1 | 5.2 |
| BSA | 236 ± 5 | 6.5 ± 0.1 | 36 | 776 ± 173 | 19.5 ± 3.9 | 40 |
| (GA)₈ | 438 ± 55 | 60.1 ± 9.0 | 7.3 | 1704 ± 248 | 95.0 ± 17 | 18 |

In Table 2, E is tensile modulus; Y is compressive modulus; (NuG2)₈ is a polyprotein made of eight tandem repeats of the protein NuG2 (Cao et al., 2008); (GB1)₈ is a polyprotein made of eight tandem repeats of the protein GB1 (Cao et al., 2007); in GRG₅RG₄R, G represents GB1 domain, and R represents the 15 residue consensus sequence of resilin (Lv et al., 2010); in NRN₄RN₄R, N represents NuG2 domain, and R represents the 15 residue consensus sequence of resilin; BSA is bovine serum albumin (Khoury et al., 2019); and (GA)₈ is a polyprotein made of eight tandem repeats of the protein GA (Alexander et al., 2009).

Although proteins are attractive building blocks to construct soft biomaterials, protein hydrogels are generally soft, making them largely inept to mimic stiff tissues (Table 1). Here we demonstrated a new DC hydrogelation approach, which combines forced-unfolding of globular proteins and chain entanglement, to enable the engineering of strong and tough protein hydrogels. On the one hand, forced-unfolding of globular proteins provides an efficient mechanism for energy dissipation, and the ability to refold allows the hydrogel to recover its mechanical properties rapidly and minimize mechanical fatigue. On the other hand, chain entanglement allows the hydrogel to achieve high stiffness. In so doing, these effects work cooperatively to allow the integration of high stiffness, high toughness, fast recovery and high compressive strength into protein hydrogels. Although the engineered protein hydrogel has a single network structure, its superb mechanical properties essentially convert a muscle-like soft biomaterial to a stiff material exhibiting mechanical properties that mimic cartilage. Our study has significantly expanded the range of mechanical properties that protein hydrogels can achieve, and thus make many existing protein hydrogels as potential candidates for developing cartilage-mimetic protein hydrogels. Given the generality of this new approach and the richness of potential protein building blocks, our study may open up an exciting new area for exploration, as well as developing new protein-based biomaterials for applications in fields ranging from cartilage repair to soft robotics and actuators.

While the disclosure has been described with reference to what are presently considered to be the preferred examples, it is to be understood that the disclosure is not limited to the disclosed examples. To the contrary, the present disclosure is intended to cover various modifications and equivalent arrangements included within the spirit and scope of the appended claims.

Citation of references herein is not an admission that such references are prior art to an embodiment of the present disclosure. Any priority document(s) and all publications, including but not limited to patents and patent applications, cited in this specification are incorporated herein by reference as if each individual publication were specifically and individually indicated to be incorporated by reference herein and as though fully set forth herein. Where a term in the present disclosure is found to be defined differently in a document incorporated herein by reference, the definition provided herein is to serve as the definition for the term.

### FULL CITATIONS FOR REFERENCES REFERRED TO IN THE DESCRIPTION

P A Alexander, Y He, Y Chen, J Orban and P N Bryan, “A minimal sequence code for switching protein structure and function” PNAS 2009, 106:50, 21149-21154.
A Bin Imran, K Esaki, H Gotoh, T Seki, K Ito, Y Sakai and Y Takeoka, “Extremely stretchable thermosensitive hydrogels by introducing slide-ring polyrotaxane cross-linkers and ionic groups into the polymer network” Nat Commun 2014, 5, 5124.
Y Cao and H Li, “Polyprotein of GB1 is an ideal artificial elastomeric protein” Nat Mater 2007, 6, 109-114.
Y Cao, R Kuske and H Li, “Direct observation of Markovian behavior of the mechanical unfolding of individual proteins” Biophys J 2008, 95, 782-788.
R H Colby, “Structure and linear viscoelasticity of flexible polymer solutions: comparison of polyelectrolyte and neutral polymer solutions” Rheol Acta 2010, 49, 425-442.
C M Elvin, A G Carr, M G Huson, J M Maxwell, R D Pearson, T Vuocolo, N E Liyou, DCC Wong, D J Merritt and N E Dixon, “Synthesis and properties of crosslinked recombinant pro-resilin” Nature 2005, 437, 999-1002.
C M Elvin, A G Brownlee, M G Huson, T A Tebb, M Kim, R E Lyons, T Vuocolo, N E Liyou, T C Hughes, JAM Ramshaw and J A Werkmeister, “The development of photochemically crosslinked native fibrinogen as a rapidly formed and mechanically strong surgical tissue sealant” Biomaterials 2009, 30, 2059-2065.
C M Elvin, T Vuocolo, A G Brownlee, L Sando, M G Huson, N E Liyou, P R Stockwell, R E Lyons, M Kim, G A Edwards, G Johnson, G A McFarland, JAM Ramshaw and J A Werkmeister, “A highly elastic tissue sealant based on photopolymerised gelatin” Biomaterials 2010, 31, 8323-8331.
D A Fancy and T Kodadek, “Chemistry for the analysis of protein—protein interactions: Rapid and efficient cross-linking triggered by long wavelength light” PNAS 1999, 96, 6020-6024; PNAS 2000, 97, 1317.
J Fang and H Li, “A facile way to tune mechanical properties of artificial elastomeric proteins-based hydrogels” Langmuir 2012, 28, 8260-8265.
J Fang, A Mehlich, N Koga, J Huang, R Koga, X Gao, C Hu, C Jin, M Rief, J Kast, D Baker and H Li, “Forced protein unfolding leads to highly elastic and tough protein hydrogels” Nat Commun 2013, 4, 2974.
D Gan, Z Wang, C Xie, X Wang, W Xing, X Ge, H Yuan, K Wang, H Tan and X Lu, “Mussel-inspired tough hydrogel with in situ nanohydroxyapatite mineralization for osteochondral defect repair” Adv Healthc Mater 2019, 8, e1901103.
J P Gong, “Why are double network hydrogels so tough?” Soft Matter 2010, 6, 2583-2590.
J P Gong, Y Katsuyama, T Kurokawa and Y Osada, “Double-network hydrogels with extremely high mechanical strength” Adv Mater 2003, 15, 1155-1158.
J Gosline, M Lillie, E Carrington, P Guerette, C Ortlepp and K Savage, “Elastic proteins: biological roles and mechanical properties” Philosophical Transactions of the Royal Society of London Series B-Biological Sciences 2002, 357, 121-132.
W C Hayes and L F Mockros, “Viscoelastic properties of human articular cartilage” Journal of Applied Physiology 1971, 31:4, 562-568.
H Higuchi, “Viscoelasticity and function of connectin/titin filaments in skinned muscle fibers”Adv Biophys 1996, 33, 159-171.
Y Jiang and R S Tuan, “Origin and function of cartilage stem/progenitor cells in osteoarthritis” Nat Rev Rheumatol 2015, 11:4, 206-212.
C P Johnson, H-Y Tang, C Carag, D W Speicher and D E Discher, “Forced unfolding of proteins within cells” Science 2007, 317, 663-666.
A J Kerin, M R Wisnom and M A Adams, “The compressive strength of articular cartilage” Proc Inst Mech Eng H 1998, 212, 273-280.
L R Khoury, J Nowitzke, K Shmilovich and I Popa, “Study of biomechanical properties of protein-based hydrogels using force-clamp rheometry”Macromolecules 2018, 51, 1441-1452.
L R Khoury and I Popa, “Chemical unfolding of protein domains induces shape change in programmed protein hydrogels” Nat Commun 2019, 10, 5439.
N Koga, R Tatsumi-Koga, G Liu, R Xiao, T B Acton, G T Montelione and D Baker, “Principles for designing ideal protein structures” Nature 2012, 491, 222-227.
H Lei, C He, C Hu, J Li, X Hu, X Hu and H Li, “Single-molecule force spectroscopy trajectories of a single protein and its polyproteins are equivalent: a direct experimental validation based on a small protein NuG2” Angew Chem Int Ed Engl 2017, 56, 6117-6121.
H Li, W A Linke, A F Oberhauser, M Carrion-Vazquez, J G Kerkvliet, H Lu, P E Marszalek and J M Fernandez, “Reverse engineering of the giant muscle protein titin” Nature 2002, 418, 998-1002.
Y Li, B Xue And Y Cao, “Synthetic protein hydrogels”ACS Macro Lett 2020, 9, 512-524.
W A Linke, V I Popov and G H Pollack, “Passive and active tension in single cardiac myofibrils” Biophys J 1994, 67, 782-792.
J Liu, C Soo, Y Tan, Z Yu, N Li, C Abell and O A Scherman, “Tough supramolecular polymer networks with extreme stretchability and fast room-temperature self-healing” Adv Mater 2017, 29, 1605325.
X L Lu and V C Mow, “Biomechanics of articular cartilage and determination of material properties” Med Sci Sports Exerc 2008, 40, 193-199.
S Lv, D M Dudek, Y Cao, M M Balamurali, J Gosline and H Li, “Designed biomaterials to mimic the mechanical properties of muscles” Nature 2010, 465, 69-73.
C W McCutchen, Lubrication of Joints, The Joints and Synovial Fluid. Vol. 10, 437 (Academic, 1978).
C L McGann, E A Levenson and K L Kiick, “Resilin-based hybrid hydrogels for cardiovascular tissue engineering” Macromolecules 2013, 214, 203-213.
N Mohan, P D Nair and Y Tabata, “A 3D biodegradable protein based matrix for cartilage tissue engineering and stem cell differentiation to cartilage” J Mater Sci Mater Med 2009, 20 Suppl 1, S49-S60.
A Nakayama, A Kakugo, J P Gong, Y Osada, M Takai, M Erata and S Kawano, “High mechanical strength double-network hydrogel with bacterial cellulose”Adv Mater 2004, 14, 1124-1128.
K Numata, N Ifuku, H Masunaga, T Hikima and T Sakai, “Silk resin with hydrated dual chemical-physical cross-links achieves high strength and toughness” Biomacromolecules 2017, 18, 1937-1946.
Y Okumura and K Ito, “The polyrotaxane gel: A topological gel by figure-of-eight cross-links” Adv Mater 2001, 13, 485-487.
B P Partlow, C W Hanna, J Rnjak-Kovacina, J E Moreau, M B Applegate, K A Burke, B Marelli, A N Mitropoulos, F G Omenetto and D L Kaplan, “Highly tunable elastomeric silk biomaterials” Adv Funct Mater 2014, 24, 4615-4624.
B P Partlow, M B Applegate, F G Omenetto and D L Kaplan, “Dityrosine cross-linking in designing biomaterials” ACS Biomater Sci Eng 2016, 2:12, 2108-2121.
M Rief, M Gautel, F Oesterhelt, J M Fernandez and H E Gaub, “Reversible unfolding of individual titin immunoglobulin domains by AFM” Science 1997, 276, 1109-1112.
D Su, M Yao, J Liu, Y Zhong, X Chen and Z Shao, “Enhancing mechanical properties of silk fibroin hydrogel through restricting the growth of beta-sheet domains” ACS Appl Mater Interfaces 2017, 9, 17489-17498.
J-Y Sun, X Zhao, WRK Illeperuma, 0 Chaudhuri, K H Oh, D J Mooney, J J Vlassak and Z Suo, “Highly stretchable and tough hydrogels” Nature 2012, 489, 133-136.
W Sun, B Xue, Q Fan, R Tao, C Wang, X Wang, Y Li, M Qin, W Wang, B Chen and Y Cao, “Molecular engineering of metal coordination interactions for strong, tough, and fast-recovery hydrogels” Sci Adv 2020, 6, eaaz9531.
D K Temple, A A Cederlund, B M Lawless, R M Aspden and D M Espino, “Viscoelastic properties of human and bovine articular cartilage: a comparison of frequency-dependent trends” BMC Musculoskelet Disord 2016, 17, 419.
LRG Treloar, The Physics of Rubber Elasticity. (Oxford University Press, USA, 1975).
S A Wainwright, W D Biggs, J D Currey and J M Gosline, Mechanical Design in Organisms. (Princeton University Press, 1982).
J Wang, L Lin, Q Cheng and L Jiang, “A strong bio-inspired layered PNIPAM-clay nanocomposite hydrogel” Angew Chem Int Ed Engl 2012, 51, 4676-4680.
A K Williamson, A C Chen, K Masuda, K., E J-M A Thonar and R L Sah, “Tensile mechanical properties of bovine articular cartilage: variations with growth and relationships to collagen network components” J Orthop Res 2003, 21, 872-880.
J Wu, P Li, C Dong, H Jiang, B Xue, X Gao, M Qin, W Wang, B Chen and Y Cao, “Rationally designed synthetic protein hydrogels with predictable mechanical properties” Nat Commun 2018, 9, 620.
L Xu, X Zhao, C Xu and N A Kotov, “Water-rich biomimetic composites with abiotic self-organizing nanofiber network” Adv Mater 2018, 30, 1703343.
L Xu, C Wang, Y Cui, A Li, Y Qiao and D Qui, “Conjoined-network rendered stiff and tough hydrogels from biogenic molecules” Sci Adv 2019, 5, eaau3442.
F Yang, J Zhao, W J Koshut, J, Watt, J C Riboh, K Gall and B J Wiley, “A synthetic hydrogel composite with the mechanical behavior and durability of cartilage” Adv Funct Mater 2020, 30, 2003451.
X Zhao, “Multi-scale multi-mechanism design of tough hydrogels: building dissipation into stretchy networks” Soft Matter 2014, 10, 672-687.

1. A method of preparing a protein hydrogel, the method comprising:
denaturing a protein in an aqueous environment to produce an aqueous composition comprising overlapping polypeptide chains;
crosslinking the polypeptide chains to produce a denatured protein hydrogel comprising a crosslinked network of entangled polypeptide chains; and
optionally at least partially renaturing the denatured protein hydrogel to produce a renatured protein hydrogel comprising a crosslinked network of entangled polypeptide chains.

2. The method of claim 1, wherein the denaturing comprises subjecting the protein to a chaotropic agent.

3. The method of claim 2, wherein the aqueous environment comprises the chaotropic agent and the method comprises introducing the protein into the aqueous environment.

4. The method of claim 2 or 3, wherein the chaotropic agent comprises guanidinium chloride.

5. The method of claim 4, wherein the concentration of the guanidium chloride in the aqueous environment is in the range of from about 6M to about 8M.

6. The method of claim 4, wherein the concentration of the guanidium chloride in the aqueous environment is about 7M.

7. The method of any one of claims 3 to 6, wherein the concentration of the protein in the aqueous environment is about 20% (w/v).

8. The method of any one of claims 1 to 7, wherein the method comprises the at least partial renaturing of the denatured protein hydrogel to produce the renatured protein hydrogel.

9. The method of claim 8, wherein the renaturing comprises equilibrating the denatured protein hydrogel in phosphate buffered saline.

10. The method of any one of claims 1 to 9, wherein the crosslinking is carried out in a mold.

11. The method of any one of claims 1 to 10, wherein the protein is a globular protein.

12. The method of any one of claims 1 to 10, wherein the protein is a tandem modular protein.

13. The method of any one of claims 1 to 12, wherein the protein has a molecular weight of greater than 33 kDa.

14. The method of any one of claims 1 to 12, wherein the protein has greater than 300 residues.

15. The method of any one of claims 1 to 14, wherein the protein is an engineered protein.

16. The method of any one of claims 1 to 15, wherein the protein comprises ferredoxin-like folds.

17. The method of any one of claims 1 to 15, wherein the protein comprises (FL)ₓ, (FL-M23C)ₓ, (NuG2)ₓ, (GB1)ₓ, (GA)ₓ, where x is the number of protein repeat units and x is at least 4, GRG₅RG₄R, N₄RN₄RNR or combinations thereof.

18. The method of any one of claims 1 to 15, wherein the protein comprises (FL)₈.

19. A protein hydrogel prepared by a method as defined in any one of claims 1 to 18.

20. A protein hydrogel comprising a crosslinked network of entangled polypeptide chains.

21. The protein hydrogel of claim 20, wherein the crosslinked network of entangled polypeptide chains comprises a combination of folded domains and unfolded domains.

22. The protein hydrogel of claim 20, wherein the crosslinked network of entangled polypeptide chains comprises about 50% folded domains.

23. The protein hydrogel of any one of claims 20 to 22, wherein the concentration of the crosslinked network of entangled polypeptide chains in the protein hydrogel is about % (w/v).

24. The protein hydrogel of any one of claims 20 to 23, wherein the crosslinked network of entangled polypeptide chains is derived from a globular protein.

25. The protein hydrogel of any one of claims 20 to 23, wherein the crosslinked network of entangled polypeptide chains is derived from a tandem modular protein.

26. The protein hydrogel of any one of claims 20 to 25, wherein the crosslinked network of entangled polypeptide chains is derived from a protein having molecular weight of greater than about 33 kDa.

27. The protein hydrogel of any one of claims 20 to 25, wherein the crosslinked network of entangled polypeptide chains is derived from a protein having greater than 300 residues.

28. The protein hydrogel of any one of claims 20 to 25, wherein the crosslinked network of entangled polypeptide chains is derived from an engineered protein.

29. The protein hydrogel of any one of claims 20 to 25, wherein the crosslinked network of entangled polypeptide chains is derived from a protein comprising ferredoxin-like folds.

30. The protein hydrogel of any one of claims 20 to 25, wherein the crosslinked network of entangled polypeptide chains is derived from a protein comprising (FL)ₓ, (FL-M23C)ₓ, (NuG2)ₓ, (GB1)ₓ, (GA)ₓ, where x is the number of protein repeat units and x is at least 4, GRG₅RG₄R, N₄RN₄RNR or combinations thereof.

31. The protein hydrogel of any one of claims 20 to 25, wherein the crosslinked network of entangled polypeptide chains is derived from a protein comprising (FL)₈.
